(19) United States
(12) Patent Application Publication
(10) Pub. No.: US 2025/0017949 A1
(43) Pub. Date: Jan. 16, 2025

(54) METHODS AND COMPOSITIONS FOR SUPPRESSING AGING-ASSOCIATED CLONAL HEMATOPOIESIS

(71) Applicant: The Jackson Laboratory, Bar Harbor, ME (US)

(72) Inventors: Jennifer Trowbridge, Bar Harbor, ME (US); Kira Young, Bar Harbor, ME (US); Jennifer SanMiguel, Bar Harbor, ME (US); Logan Schwartz, Bar Harbor, ME (US)

(73) Assignee: The Jackson Laboratory, Bar Harbor, ME (US)

(21) Appl. No.: 18/706,942

(22) PCT Filed: Nov. 3, 2022
(86) PCT No.: PCT/US2022/048767

§ 371 Date: May 2, 2024

Related U.S. Application Data
(60) Provisional application No. 63/274,994, filed on Nov. 3, 2021.

(51) Int. Cl. A61K 31/66; A61P 7/00; C07K 16/24; C07K 16/28

(52) U.S. Cl. CPC A61K 31/66; A61P 7/00; C07K 16/248; C07K 16/2878

(57) ABSTRACT

The present disclosure is directed to methods of treating clonal hematopoiesis in a subject thereof, comprising administering to the subject a mitochondria-targeted antioxidant in an amount effective to suppress clonal hematopoiesis in the subject, relative to an untreated control where the mitochondria-targeted antioxidant is a shortened form of the antioxidant ubiquinol with triphenylphosphonium.

### RELATED APPLICATION

This application claims the benefit under 35 U.S.C. § 119(e) of U.S. provisional application No. 63/274,994, filed Nov. 3, 2021, which is incorporated by reference herein in its entirety.

### GOVERNMENT LICENSE RIGHTS

This invention was made with government support under Grant No. NIH R01 DK118072 and Grant No. NIH R01 AG069010 awarded by National Institutes of Health. The government has certain rights in the invention.

### BACKGROUND

During human aging, somatic mutations accumulate in the long-lived hematopoietic stem cell (HSC) pool. HSCs with certain somatic mutations, most commonly in the DNA methyltransferase DNMT3A, undergo positive selection leading to clonal HSC expansion, which has been termed clonal hematopoiesis (CH). While CH is a benign condition, individuals with CH have increased risk of developing blood cancers (e.g., hematologic malignancy), cardiovascular disease, and overall have increased all-cause mortality. Factors in the aging bone marrow microenvironment are a major cause of impaired function and myeloid-biased hematopoiesis from non-mutant HSCs.

### SUMMARY

Understanding the variables that lead to and/or promote clonal expansion and further transformation are important for identifying individuals at risk and for developing improved biomarkers and therapeutic strategies to combat aberrant clonal expansion and transformation and its associated diseases. To develop interventions to stop or slow mutant HSC expansion in aging individuals, a key gap in current knowledge is a lack of understanding of the mechanisms by which HSCs with DNMT3A or other frequent mutations possess a selective advantage.

Some aspects of the present disclosure provide a method of treating clonal hematopoiesis in a subject in need thereof, comprising administering to the subject a mitochondria-targeted antioxidant in an amount effective to suppress clonal hematopoiesis in the subject, relative to an untreated control.

In some embodiments, the subject exhibits signs of defects in mitochondrial metabolism.

In some embodiments, administration of the mitochondria-targeted antioxidant improves mitochondrial metabolism in the subject, relative to an untreated control. For example, administration of the mitochondria-targeted antioxidant may improve mitochondrial metabolism in the subject by at least 30%, at least 40%, or at least 50%, relative to an untreated control.

In some embodiments, the mitochondria-targeted antioxidant is a shortened form of the antioxidant ubiquinol with triphenylphosphonium (e.g., MitoQ®).

In some embodiments, the method further comprises identifying the subject as exhibiting a sign of clonal hematopoiesis. The sign of clonal hematopoiesis may be, for example, a defect in mitochondrial metabolism.

Other aspects of the present disclosure provide a method of treating clonal hematopoiesis in a subject in need thereof, comprising administering to the subject a TNF signaling inhibitor in an amount effective to suppress clonal hematopoiesis in the subject, relative to an untreated control.

In some embodiments, the subject exhibits signs of elevated TNF signaling.

In some embodiments, administration of the TNF signaling inhibitor specifically reduces TNF signaling through TNFR1 in the subject.

In some embodiments, the TNF signaling inhibitor is an antibody that specifically binds to TNFR1.

In some embodiments, the method further comprises identifying the subject as exhibiting a sign of clonal hematopoiesis.

In some embodiments, the sign of clonal hematopoiesis is elevated TNF signaling.

Yet other aspects of the present disclosure provide a method of treating clonal hematopoiesis in a subject in need thereof, comprising administering to the subject a OSM signaling inhibitor in an amount effective to suppress clonal hematopoiesis in the subject, relative to an untreated control.

In some embodiments, the subject exhibits signs of elevated OSM signaling.

In some embodiments, administration of the OSM signaling inhibitor reduces OSM signaling in the subject.

In some embodiments, the OSM signaling inhibitor is an antibody that specifically binds to OSM or OSMR.

In some embodiments, the method further comprises identifying the subject as exhibiting a sign of clonal hematopoiesis.

In some embodiments, the sign of clonal hematopoiesis is elevated OSM signaling.

In some embodiments, the subject is at risk of blood cancer.

In some embodiments, the subject is at risk of cardiovascular disease.

### BRIEF DESCRIPTION OF THE DRAWINGS

FIG. 1: Loss of Igf1 in the bone marrow microenvironment is sufficient to cause Dnmt3a-mutant hematopoietic stem cell (HSC) expansion. Frequency of donor-derived HSCs at 24 weeks post-transplant of MxCre control or Dnmt3a-mutant (D3a) hematopoietic cells into Igf1 wild type (Igf1 +/+) or Igf1 conditional knockout (Igf1 −/−) recipient animals.

FIG. 2: Inhibition of mTOR is sufficient to cause Dnmt3a-mutant hematopoietic stem cell (HSC) expansion. Frequency of donor-derived HSCs at 24 weeks post-transplant of MxCre control or Dnmt3a-mutant (D3a) hematopoietic cells into wild-type recipient animals that were treated post-transplant with rapamycin-supplemented diet (eRAPA) or control (CNT) diet.

FIG. 3: Treatment with MitoQ decreases myeloid differentiation of hematopoietic stem and progenitor cells in vitro. Number of myeloid colonies produced from wild-type MxCre control or Dnmt3a-mutant (D3a) hematopoietic stem and progenitor cells in media containing MitoQ or vehicle (DMSO).

FIG. 4: Treatment with MitoQ decreases contribution of Dnmt3a-mutant cells to mature hematopoiesis in vivo. Frequency of donor-derived cells produced from wild-type (MxCre) control or Dnmt3a-mutant (D3a) hematopoietic stem cells post-transplantation and in vivo treatment with MitoQ or vehicle (PBS).

FIGS. 5A-5I: Dnmt3a^{R878H/+} HSCs engage a TNFα-induced program in the aged BM microenvironment that is conserved in human DNMT3A-mutant clonal hematopoiesis. (FIG. 5A) Schematic of experimental design to compare Mx-Cre control and Dnmt3a^{R878H/+} (R878H/+) engraftment in young (2-4 mo) and aged (13-15 mo) recipient mice. (FIG. 5B) Frequency of donor (CD45.2⁺) cells in peripheral blood (PB) of recipient mice post-transplant. Significance calculated using two-way ANOVA with Tukey's multiple comparisons test. (FIG. 5C) Frequency of donor cells in bone marrow (BM) of recipient mice. Significance calculated using one-way ANOVA with Bonferroni's multiple comparisons test. (FIG. 5D) Frequency of HSCs and MPPs in donor-derived BM cells. Significance calculated using two-way ANOVA with Fisher's LSD. (FIG. 5E) Frequency of MPP^{Mk/E}, MPP^{G/M} and MPP^{Ly} in donor-derived BM cells. Significance calculated using two-way ANOVA with Fisher's LSD. (FIG. 5F) Volcano plots with significantly differentially expressed genes (FDR<0.5, log FC>2) within shaded boxes (n=2-4 biological replicates). (FIG. 5G) Enrichment of hallmark gene sets (left panel) and predicted upstream regulators (right panel) in control vs. Dnmt3a^{R878H/+} HSCs in aged recipient mice. (FIG. 5H) Hallmark TNF pathway enrichment across stem and progenitor populations between human DNMT3AR882H vs control CD34+ cells. (FIG. 5I) Overlap of differentially expressed genes in DNMT3AR882H vs control CD34+ cells and R878H/+ vs aged mouse HSCs. (FIGS. 5B-5E) Dots represent individual recipient mice, boxes show 25 to 75^{th} percentile, line is median, whiskers show min to max. *P<0.05, **P<0.01, ***P<0.001.

FIGS. 6A-6I: Dnmt3a-mutant HSCs maintain self-renewal and generate B lymphoid cells following TNF stimulation. (FIG. 6A) Schematic of experimental design to test response of Mx-Cre control and Dnmt3a^{R878H/+} HSCs to recombinant TNFα ex vivo under growth conditions that favor HSC expansion. (FIG. 6B) Normalized frequency of donor-derived cells in PB of recipient mice post-transplant. Significance calculated using mixed-effects model with Fisher's LSD. (FIG. 6C) Representative flow cytometry plots showing B cell and myeloid cell frequencies in donor derived PB at 4 weeks post-transplant. (FIG. 6D) Frequency of myeloid (left) and B cells (right) in donor derived PB at 4 weeks post-transplant. Significance calculated using two-way ANOVA with Sidak's multiple comparison's test. (FIG. 6E) Schematic of experimental design to test TNFα response of Fgd5-Cre^{ERT} control vs. Fgd5-Cre^{ERT} Dnmt3a^{R878H/+} HSCs, and germline Dnmt3a^{+/+} vs. Dnmt3a^{+/-} HSCs. (FIGS. 6F-6G) Viable cell counts after 7 days of culture. Significance calculated using Brown-Forsythe and Welch ANOVA with Welch's correction. (FIG. 6H) Frequency of donor-derived cells in PB of recipient mice post-transplant. Significance calculated using mixed-effects model with Fisher's LSD. (FIG. 6I) Frequency of myeloid (left) and B cells (right) in donor derived PB at 4 weeks post-transplant. Significance calculated using one-way ANOVA with Fisher's LSD. (FIGS. 6B, 6D, and 6F-6I) Dots represent individual recipient mice, boxes show 25 to 75^{th} percentile, line is median, whiskers show min to max. *P<0.05, **P<0.01, ***P<0.001.

FIGS. 7A-7H: TNFR1 is required for Dnmt3a^{R878H/+} HSC self-renewal while TNFR2 regulates lymphoid cell production. (FIG. 7A) Schematic of experimental design to test competitive, serial transplant of Mx-Cre control, Dnmt3a^{R878H/+}(R878H/+), Dnmt3a^{R878H/+} Tnfrsf1a^{-/-} (R878H/+; TNFR1KO) and Dnmt3a^{R878H/+} Tnfrsf1b^{-/-} (R878H/+; TNFR2KO) in aged (>12 mo) recipient mice. (FIG. 7B) Frequency of donor cells in PB (left) and BM (right) of recipient mice at 20 weeks post-secondary transplant. Significance was calculated using Brown-Forsythe and Welch ANOVA with Welch's correction. (FIG. 7C) Representative flow cytometry plots showing B cell and myeloid cell frequencies in donor derived PB. (FIG. 7D) Frequency of B cells (left), T cells (center) and myeloid cells (right) in donor derived PB at 20 weeks post-secondary transplant. Significance was calculated using one-way ANOVA with Tukey's multiple comparisons test. (FIG. 7E) Experimental schematic of transplant experiment with etanercept treatment. (FIG. 7F) Frequency of control or R878H/+ donor cells in PB pre- and post-etanercept treatment. Significance was calculated using two-way ANOVA with Fisher's LSD. (FIG. 7G) Frequency of control or R878H/+ donor cells in BM post-etanercept or vehicle treatment. Significance was calculated using two-way ANOVA with Fisher's LSD. (FIG. 7H) Frequency of HSC, MPP^{Mk/E}, MPP^{G/M}, and MPP^{Ly} populations in control or R878H/+ BM. Significance was calculated using two-way ANOVA with Fisher's LSD. (FIGS. 7B and 7D) Dots represent individual recipient mice, boxes show 25 to 75^{th} percentile, line is median, whiskers show min to max. (FIG. 7F) Dots represent mean across biological replicates (n=4). (FIGS. 7G and 7H) Bars represent mean+/− SEM (n=4). *P<0.05, **P<0.01, ***P<0.001, ****P<0.0001.

FIGS. 8A-8G: TNFR1 and TNFR2 engage distinct transcriptional programs in Dnmt3a^{R878H/+} HSCs. (FIG. 8A) Experimental schematic for single cell (sc) RNA-seq. Data was collected from independent biological replicates of Mx-Cre control (n=3), Dnmt3a^{R878H/+} (n=4), Dnmt3a^{R878H/+} Tnfrs1a^{-/-} (n=3) and Dnmt3a^{R878H/+} Tnfrs1b^{-/-} (n 4). (FIG. 8B) UMAP projection of combined data identifying 22 cell clusters. (FIG. 8C) Pseudotime visualization showing predicted differentiation trajectories from HSCs to erythroid (Ery), megakaryocyte (Mk), myeloid (My), B cell (B) and dendritic cell (DC) lineages in each genotype pool. (FIG. 8D) TNF signaling enrichment score in R878H/+ vs. control cell clusters. (FIG. 8E) Heatmap representing fold change in expression of Tnf, Tnfrsf1a (TNFR1), Tnfrsf1b (TNFR2), and downstream TNF-regulated genes comparing R878H/+ vs. control HSCs, R878H/+; R1KO vs. R878H/+ HSCs, and R878H/+; R2KO vs. R878H/+ HSCs. “#” indicate loci hypomethylated in R878H/+ vs. control HSCs. (FIG. 8F) Venn diagrams of overlap between upregulated genes (top) and downregulated genes (bottom) in R878H/+; R1KO and R878H/+; R2KO HSCs compared to R878H/+ HSCs. From each comparison, unique gene lists were used to determine gene signature enrichment. (FIG. 8G) The working model was created with BioRender.com. TNFα-TNFR1 signaling dictates Dnmt3a-mutant HSC self-renewal whereas TNFα-TNFR2 signaling promotes lymphoid lineage cell production. Decline in TNFα-TNFR2 signaling results in unrestrained production of Dnmt3a-mutant myeloid cells.

FIGS. 9A-9B: Dnmt3a-mutant hematopoietic stem and progenitor cells (HSPCs) have a selective advantage over wild-type HSPCs in an aged bone marrow microenvironment. (FIG. 9A) Experiment design. (FIG. 9B) Frequency (left) and total number (right) of wild-type (+/+) and Dnmt3a-mutant (R878H) HSPCs in young (Y) or aged (A) recipient mice at 16 weeks post-transplant.

FIGS. 10A-10G: OSM signaling is increased in Dnmt3a-mutant hematopoietic stem cells (HSCs) in an aged bone marrow microenvironment. (FIG. 10A) Experiment design. (FIG. 10B) Volcano plot of differentially expressed genes between Dnmt3a-mutant (R878H) vs. wild-type (+/+) HSCs in young recipients (left panel) and aged recipients (right panel). Significantly differentially expressed genes (FDR<0.05) are shown by green and red colored dots. (FIG. 10C) Ingenuity Pathway Analysis prediction of upstream factors that would produce the differential gene expression pattern observed in aged recipients shown in FIG. 10B. (FIG. 10D) Gene set enrichment analysis of OSM signaling signature in Dnmt3a-mutant (R878H) vs. wild-type (+/+) HSCs specifically in aged recipient mice. (FIG. 10E) OSM levels in bone marrow fluid of young, middle-aged and old C57BL/6 wild-type mice assessed by ELISA. (FIG. 10F) Schematic of OSM-OSMR signaling pathway with differentially expressed genes in Dnmt3a-mutant vs. control HSCs colored in green (increased) or orange (decreased). (FIG. 10G) Phosphorylation of Stat3 (left panel) and Stat5 (right panel) in wild-type or Dnmt3a-mutant HSPCs in response to recombinant OSM stimulation for 30 minutes.

FIGS. 11A-11C: OSM favors growth of Dnmt3a-mutant hematopoietic stem cells (HSCs). (FIG. 11A) Experiment design. Contribution of wild-type (WT F1) and competitor Dnmt3a-mutant (R878H/+) cells to peripheral blood (FIG. 11B) and bone marrow (FIG. 11C) of recipient mice after culture with or without recombinant OSM.

FIGS. 12A-12B: Loss of OSM-OSMR signaling causes mature myeloid cell differentiation from Dnmt3a-mutant HSPCs. (FIG. 12A) Experiment design. (FIG. 12B) Proportion of control (+/+), Dnmt3a-mutant (R878H/+) and Dnmt3a-mutant OSMR−/− (R878H/+ Osmr−/−) mature myeloid (CD11b+ Gr1+) cells produced from HSPCs.

### DETAILED DESCRIPTION

### I. Clonal Hematopoiesis

Provided herein, in some aspects, are methods and compositions for treating (e.g., preventing or suppressing) clonal hematopoiesis. Hematopoietic stem cells (HSCs) are a population of resident bone marrow (BM) cells. HSCs are capable of self-renewal and have the capacity to reconstitute all types of blood cells, including white blood cells, red blood cells, and platelets. When somatic mutations accumulate in HSCs and undergo positive selection this leads to clonal HSC expansion called clonal hematopoiesis (CH). As a result, the blood cells generated by CH will all have the same genetic mutation and a different genetic pattern than the pre-existing blood cells. Most people with CH do not show symptoms of disease. However, people with CH have an increased risk of developing cardiovascular disease and blood cancers, such as myelodysplastic syndrome and acute myeloid leukemia. No single cause of CH has been identified, characteristics that can increase risk of developing CH include age, smoking and being male and white. Additionally, radiation therapy and some chemotherapies may be linked to CH. Aging is a main driving factor of CH, the percentage of people under the age of 50 years that have CH somatic mutations is only 1%, however individuals older than 65 years are at a 10% risk, which jumps about 20% of those older than 90 years old. The most frequently mutated genes include DNMT3A, TET2, JAK2, and ASXL1.

### A. Mitochondrial Metabolism

In some embodiments, a method of treating clonal hematopoiesis includes modulating mitochondrial metabolism in a subject. Cellular aging can be considered in both chronological age and physiological age. Chronological age is the actual age of the organism, whereas physiological age reflects an age-linked performance characteristic. Cells of the same chronological age can therefore have different physiological ages. For example, mouse HSCs may be heterogeneous with regard to their physiological age. Cell-intrinsic mechanisms underlying HSC aging, include increased reactive oxygen species (ROS) production, and mitochondrial dysfunction such as mitochondrial membrane potential (MMP). MMP directly determines both the rate of transcription and the nature of gene expression of HSCs. Insulin-Like Growth Factor 1 (IGF1) in the aging bone marrow microenvironment is a major factor causing impaired function and myeloid-biased hematopoiesis from non-mutant HSCs. Decline in IGF1 in the bone marrow microenvironment promotes Dnmt3a-mutant CH by conferring a selective advantage of Dnmt3a-mutant HSCs over wild-type HSCs.

Mitochondria metabolism declines with age. Mitochondrial metabolism includes pathways that generate adenosine triphosphate (ATP) to drive intracellular energetic reactions and produce the building blocks necessary for macromolecule synthesis. In some embodiments, a method provided herein includes modulating mitochondrial metabolism in a subject who exhibits signs (or symptoms) of defects in mitochondrial metabolism. Signs of defects in mitochondrial metabolism in a subject may encompass, for example, insufficient ATP synthesis.

In some embodiments, the defects in mitochondrial metabolism may be selected from one or more of the following: insufficient ATP synthesis, defects in mitochondrial transport, defects of substrate utilization (e.g., PDH deficiency), defects of the Krebs cycle, defects of oxidation-phosphorylation coupling, or abnormalities of the respiratory chain (e.g., defects in complex I, defects in complex II, defects in complex III, defects in complex IV, defect in complex V, or coenzyme Q10 (CoQ10) deficiency). In some embodiments, the defects in mitochondrial metabolism is insufficient ATP synthesis.

Mitochondrial targeting is a strategy that addresses pathologies originating from mitochondrial dysfunction. In some embodiments, a mitochondria-targeted antioxidant is administered to a subject in an amount effective to suppress clonal hematopoiesis. In some embodiments, a mitochondria-targeted antioxidant is administered to a subject in an amount effective to suppress clonal hematopoiesis, relative to an untreated control hematopoiesis. In some embodiments, clonal hematopoiesis is suppressed by about 15-100 (15-100, 15-75, 15-50, 15-25, 25-100, 25-50, 25-75, 50-100, 50-75, or 75-100) % relative to an untreated control hematopoiesis. In some embodiments, clonal hematopoiesis is suppressed by about 15%, about 25%, about 50%, about 75%, about 100% relative to an untreated control hematopoiesis.

Antioxidants reduce excessive levels of highly reactive oxidants such as reactive oxygen species (ROS). The ROS group consists of unstable radicals (superoxide anion, hydroxyl anion and singlet oxygen, including derivatives thereof) and hydrogen peroxide. Therefore, mitochondria-targeted antioxidants are therapies that specifically quench mitochondrial reactive oxygen species (ROS). Administration of the mitochondrially targeted antioxidant mitoquinone mesylate (MitoQ) can be used to reverse metabolic conditions that favor Dnmt3a-mutant selective advantage over wild-type. Mitochondria produce important enzymes, such as CoQ10. CoQ10 allows mitochondria to create cellular energy and neutralize harmful free radicals. With aging, mitochondria produce less CoQ10 enzyme, approximately 10% less every decade. Drugs such as MitoQ deliver CoQ10 support to mitochondria and increase wild-type HSC function in aging. However, CH mutant stem cells have a different response to this drug intervention. Administering MitoQ reduces the burden of Dnmt3a-mutant CH. Therefore, MitoQ administration to Dnmt3a-mutant mice can be used as a therapeutic to reduce clonal hematopoiesis and its associated complications, including blood cancer development and cardiovascular disease. In some embodiments, the method comprises administering to a subject a mitochondria-targeted antioxidant. Examples of mitochondria-targeted antioxidants include, but are not limited to, MitoQ®, MitoVitE, MitoPBN, MitoPeroxidase, MITO-Porter, SkQs (e.g., SkQ1, SkQR1, SkQTR1 and SkQT1), Mito-Vit-E, mito-TEMPO, SS peptides (e.g., peptide SS-31), and XJB-5-131.

In some embodiments, the mitochondria-targeted antioxidant is a shortened form of the antioxidant ubiquinol with triphenylphosphonium (e.g., MitoQ®). In some embodiments, MitoQ® is PubChem CID 11388331, as shown in Formula I.

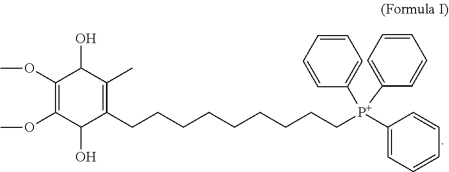 [SMILES: COC1=C(OC)C(O)C(CCCCCCCCC[P+](c2ccccc2)(c2ccccc2)c2ccccc2)=C(C)C1O]

In some embodiments, the mitochondria-targeted antioxidant (e.g., MitoQ®) is administered at a daily dose of 500-1200 (e.g., 500-1200, 500-1000, 500-750, 750-1200, 750-1000, 1000-1200) mg. In some embodiments, the mitochondria-targeted antioxidant (e.g., MitoQ®) is administered at a daily dose of about 500, about 750, about 1000, about 1200 mg. In some embodiments, the mitochondria-targeted antioxidant (e.g., MitoQ®) is administered at a daily dose of about 10 (e.g., 8, 10, 12, 15, 20) mg. In some embodiments, the mitochondria-targeted antioxidant (e.g., MitoQ®) is administered at a daily dose of about 8, about 10, about 12, about 15, about 20. In some embodiments, the mitochondria-targeted antioxidant (e.g., MitoQ®) is administered at a daily dose of about 10 mg. In some embodiments, the mitochondria-targeted antioxidant (e.g., MitoQ®) is administered orally.

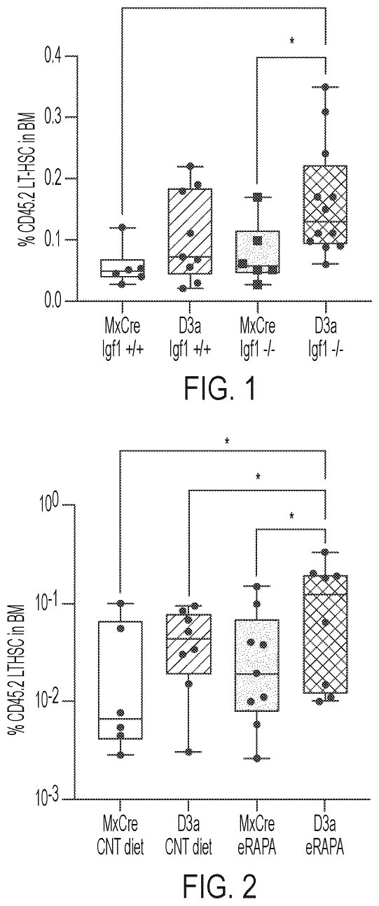

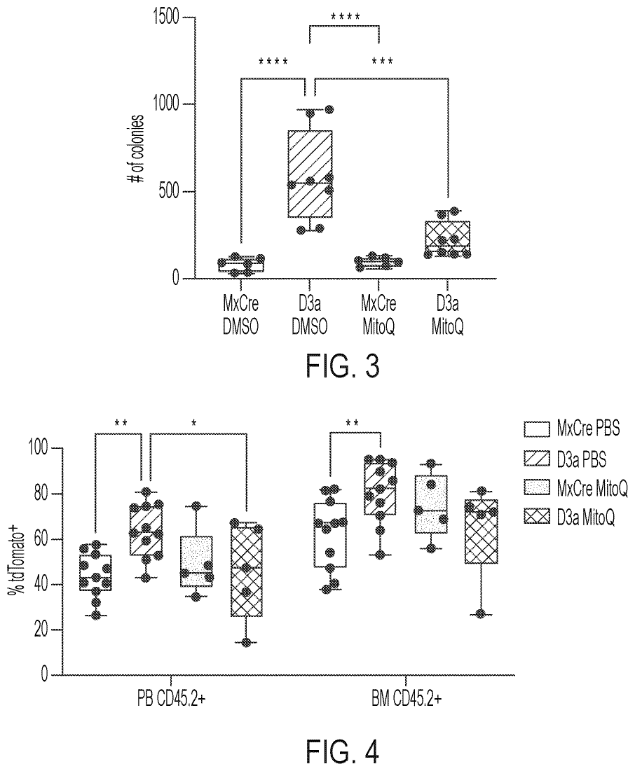

As discussed above, clonal hematopoiesis (CH) is an aging-associated condition driven by somatic mutations in long-lived hematopoietic stem cells (HSCs), the most common of which is in the DNA methyltransferase DNMT3A gene. Individuals with CH have increased risk of developing blood cancers, cardiovascular disease, and overall have increased all-cause mortality. Understanding the variables that promote CH will lead to development of improved biomarkers and therapeutic targets to prevent CH and its associated diseases. Using a mouse model of DNMT3A^{R882H} (mouse Dnmt3a^{R878H}), we have found that transplant of Dnmt3a^{R878H} cells into aged recipients results in Dnmt3a-mutant HSC expansion. Recent work (Young et al., Cell Stem Cell 2021) demonstrated that decline in Insulin-Like Growth Factor 1 (IGF1) in the aging bone marrow microenvironment is a major factor causing impaired function and myeloid-biased hematopoiesis from non-mutant HSCs. Here, the extent to which decline in IGF1 in the bone marrow microenvironment may promote Dnmt3a-mutant clonal hematopoiesis was tested. By transplantation of wild-type and Dnmt3a^{R878H} HSCs into IGF1 conditional knockout (cKO) recipient mice, an increased selective advantage of Dnmt3a-mutant HSCs over wild-type HSCs was observed (FIG. 1). A similar phenotype was observed with pharmacological inhibition of IGF1-mTOR signaling using rapamycin treatment of young mice (FIG. 2), suggesting that metabolic regulation underlies this phenotype. In attempt to reverse metabolic mechanisms that favor Dnmt3a-mutant selective advantage, the mitochondrially targeted antioxidant mitoquinol (MitoQ®) was used. Ex vivo treatment with MitoQ® reduced myeloid differentiation of Dnmt3a-mutant HSCs (FIG. 3). In vivo treatment of mice with MitoQ® resulted in impaired differentiation and mature cell production from Dnmt3a-mutant HSCs (FIG. 4). Together, this suggests that MitoQ® can both improve function of wild-type HSCs while also impairing function of Dnmt3a-mutant HSCs, tipping the balance to suppress clonal hematopoiesis.

In some embodiments, identifying the subject as exhibiting a sign of clonal hematopoiesis includes, but is not limited to, analyzing the DNA collected from a blood sample for somatic mutations in genes associated with CH, identifying a subject with a defect in mitochondrial metabolism, has elevated TNF signaling, or elevated OSM signaling, exhibiting characteristics that can increase risk of developing CH.

In some embodiments, identifying the subject as exhibiting a sign of clonal hematopoiesis includes analyzing the DNA collected from a blood sample for somatic mutations in genes associated with CH. These mutations are associated with clonal hematopoietic expansion and malignancies, mainly DNMT3A, TET2, and ASXL1, and other frequently mutated genes TP53, JAK2, SF3B1. In some embodiments, the gene associated with CH is DNMT3A.

In some embodiments, identifying the subject as exhibiting a sign of clonal hematopoiesis includes identifying a subject with a defect in mitochondrial metabolism, has elevated TNF signaling, or elevated OSM signaling.

In some embodiments, identifying the subject as exhibiting a sign of clonal hematopoiesis includes exhibiting characteristics that can increase risk of developing CH. In some embodiments, a cause of CH may be selected from, but not limited to, age, smoking, being male, being Caucasian, currently undergoing or has undergone radiation therapy and/or chemotherapy. In some embodiments. exhibiting characteristics that can increase risk of developing CH may be being over the age of 45-100 (e.g., 45-100, 45-90, 45-80, 45-70, 45-60, 45-55, 45-50, 50-100, 50-90, 50-80, 50-70, 50-60, 50-55, 55-100, 55-90, 55-80, 55-70, 55-60, 60-100, 60-90, 60-80, 60-70, 70-100, 70-90, 70-80, 80-100, 80-90, or 90-100) years old. In some embodiments. exhibiting characteristics that can increase risk of developing CH may be being over the age of about 45, 50, 55, 60, 70, 80, 90, 100. In some embodiments. exhibiting characteristics that can increase risk of developing CH may be being over the age of about 50 years old.

In some embodiments, a suppression of CH in the subject comprises a reduction of CH relative to an untreated control. For example, a suppression of CH in vitro may be represented by about a 20 (e.g., 15, 20, 25, 30, 35, 40)% reduction in number of myeloid colonies produced from Dnmt3a-mutant hematopoietic stem and progenitor cells in media containing MitoQ (experimental) or vehicle/DMSO (untreated control). In some embodiments, a suppression of CH in vitro may be represented by about a 15%, 20%, 25%, 30%, 35%, or 40% reduction in number of myeloid colonies produced from Dnmt3a-mutant hematopoietic stem and progenitor cells in media containing mitochondria-targeted antioxidant (e.g., MitoQ®) (experimental) or vehicle/DMSO (untreated control). In some embodiments, a suppression of CH in vitro may be represented by about a 20% reduction in number of myeloid colonies produced from Dnmt3a-mutant hematopoietic stem and progenitor cells in media containing mitochondria-targeted antioxidant (e.g., MitoQ®) (experimental) or vehicle/DMSO (untreated control).

In vivo, a suppression of CH in the subject may be represented by about a 15 (e.g., 15, 20, 25, 30, 35, 40)% reduction in the frequency of donor-derived cells produced from Dnmt3a-mutant (D3a) hematopoietic stem cells post-transplantation and in vivo treatment with mitochondria-targeted antioxidant (e.g., MitoQ®) (experimental) or vehicle/PBS (untreated control). In some embodiments, a suppression of CH in vivo may be represented by about a 15%, 20%, 25%, 30%, 35%, or 40% reduction in the frequency of donor-derived cells produced from Dnmt3a-mutant (D3a) hematopoietic stem cells post-transplantation and in vivo treatment with mitochondria-targeted antioxidant (e.g., MitoQ®) (experimental) or vehicle/PBS (untreated control). In some embodiments, a suppression of CH in vivo may be represented by about a 15% reduction in the frequency of donor-derived cells produced from Dnmt3a-mutant (D3a) hematopoietic stem cells post-transplantation and in vivo treatment with mitochondria-targeted antioxidant (e.g., MitoQ®) (experimental) or vehicle/PBS (untreated control).

In some embodiments, improves mitochondrial metabolism in the subject comprises a suppression of CH, reduction in myeloid colonies or reduction in the frequency of donor-derived cells produced from HSCs post-transplantation compared to untreated controls. In some embodiments, an improvement in mitochondrial metabolism may be a 15-100 (e.g., 15-100, 15-90, 15-80, 15-70, 15-60, 15-50, 15-40, 15-30, 15-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90. 50-80, 50-70, 50-60, 60-100, 60-90, 60-80, 60-70, 70-100, 70-90, 70-80, 80-100, 80-90, 90-100) % reduction in myeloid colonies. In some embodiments, an improvement in mitochondrial metabolism may be about 15%, about 20%, about 30%, about 40%, about 50%, about 60%, about 70%, about 80%, about 90%, or about 100% reduction in myeloid colonies. In some embodiments, an improvement in mitochondrial metabolism may be about 20% reduction in myeloid colonies.

In some embodiments, an improvement in mitochondrial metabolism may be a 15-100 (e.g., 15-100, 15-90, 15-80, 15-70, 15-60, 15-50, 15-40, 15-30, 15-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90. 50-80, 50-70, 50-60, 60-100, 60-90, 60-80, 60-70, 70-100, 70-90, 70-80, 80-100, 80-90, 90-100) % reduction in the frequency of donor-derived cells produced from HSCs post-transplantation compared to untreated controls. In some embodiments, an improvement in mitochondrial metabolism may be about 15%, about 20%, about 30%, about 40%, about 50%, about 60%, about 70%, about 80%, about 90%, or about 100% reduction in the frequency of donor-derived cells produced from HSCs post-transplantation compared to untreated controls. In some embodiments, an improvement in mitochondrial metabolism may be about 15% reduction in the frequency of donor-derived cells produced from HSCs post-transplantation compared to untreated controls.

The term “a shortened form of the antioxidant ubiquinol with triphenylphosphonium (e.g., MitoQ®)” is defined as a mitochondrially targeted antioxidant compound comprising a lipophilic cation covalently coupled to an antioxidant moiety. The lipophilic cation is the triphenylphosphonium cation. Other lipophilic cations which may be covalently coupled to antioxidants include tribenzyl or triphenyl ammonium cation or the tribenzyl or a substituted triphenyl phosphonium cation.

### B. TNF Signaling

In some embodiments, a method of treating clonal hematopoiesis includes modulating TNF signaling (e.g., modulating TNF signaling). Tumor necrosis factor (TNF) is a 26 kDa transmembrane protein that assembles into a homotrimeric molecule (tmTNF) that can be proteolytically cleaved resulting in soluble TNF homotrimers (sTNF; 51 kDa). TNF binds to two transmembrane receptors TNFR1 and TNFR2. Both TNF receptors contain four cysteine-rich domains (CRD), and a preligand binding assembly domain (PLAD). TNFR1 is expressed on almost all nucleated cells, whereas the expression of TNFR2 is more restricted.

As a cytokine, TNF plays important roles in cell survival, proliferation, differentiation and death. Immune cells activated in response to infection or tissue damage secrete TNF. TNF is a key regulatory component of the immune system that is essential to promote tissue homeostasis and fight infections. Pathology, such as chronic inflammation and tissue damage, occur when TNF is not regulated properly. Therapeutics have been developed to counteract the pathology associated with dysregulation of TNF. There are currently five structurally different anti-TNF drugs approved for clinical use, infliximab (Remicade), adalimumab (Humira), certolizumab pegol (Cimzia), golimumab (Simponi), and etanercept (Enbrel). Limitations of these anti-TNF therapeutics include, restricted responsiveness, and severe side-effects, such as development of autoimmune diseases and lymphomas. These limitations depend on TNF's pleiotropic biological functions via two distinct TNF receptors TNFR1 and TNFR2. Genetic deletion of TNFR1 in animal models of disease leads to reduced disease. Comparatively, specific deletion of TNFR2 exacerbates disease.

Following binding of either sTNF or tmTNF to TNFR1, several other proteins are recruited to the receptor to form the TNFR1 complex. These proteins include TNF receptor 1 associated protein with death domain (TRADD), the receptor interacting protein kinase 1 (RIP1), TNF receptor associated factor 2 (TRAF2), and the cellular inhibitor of apoptosis proteins (cIAPs) 1 and 2. The TNFR1 complex can act through the canonical transcription factor nuclear factor kappa B (NFkB) pathway or the p38 MAP kinase/JNK pathway or the apoptotic or necroptotic pathways.

In the canonical NFkB pathway, after the initial assembly, other proteins are recruited through a docking platform for the linear ubiquitin assembly complex (LUBAC). These newly recruited proteins include the inhibitor of kappa B kinases (IKK) complex and the MAP3K transforming growth factor-β (TGFβ)-activated kinase-1 (TAK1). TAK1 binds to TNFR1 complex via the adapter protein TAK1-binding protein-2 (TAB2). Components of the IKK complex, IKKb and NEMO are modified by TAK1 and LUBAC. These modifications lead to the degradation of inhibitor of kappa B-alpha (IkBa), which allows for the dissociation of IkB from NFkB. Subsequently, NFkB translocates to the nucleus and modifies transcription of NFkB-regulated targets.

Next to the canonical NFkB pathway, the TNFR1 signaling complex I can bind and activate distinct MAP kinase kinases (MKK) to activate p38 and JNK which leads to the nuclear localization of c-Jun and modification of gene transcription.

To activate the apoptotic pathway, the internalization of the signaling complex leads to the dissociation of TRAF2 and the cIAPs. Subsequently, adaptor proteins are recruited to the TNFR1 signaling complex to form the death inducing signaling complex (DISC). These adaptor proteins include Fas associated death domain protein (FADD) and procaspase 8. Within DISC procaspase 8 is activated by autocatalytic cleavage resulting in activation of the effector caspase cascade that induces apoptosis.

To activate necroptosis pathway, a necrosome is formed when caspase 8 is absent or inactivated. To accomplish this the protein RIPK1 recruits and activates RIPK3. Mixed lineage kinase domain-like protein (MLKL) is a constitutive binding partner of RIPK3 and therefore incorporated in the necrosome. When MLKL is phosphorylated, it results in a conformational change, recruitment to the plasma membrane and execution of necroptosis.

In contract to TNFR1, which can bind either sTNF or tmTNF, TNFR2 is only activated by tmTNF. When TNFR2 is activated and forms the TNFR2 signaling complex the following proteins are recruited, TRAF2, cIAP1/cIAP2, and HOIP, a LUBAC component. The TNFR2 signaling complex can activate the canonical NFkB activation via IKKb and the non-canonical NFkB pathway. Activation of the non-canonical NFkB pathway requires the kinase NIK which phosphorylates and activates IKKa leading to nuclear translocation of p52/RelB NFkB heterodimers. The TNFR2 complex can also activate the p38 MAP kinase/JNK pathway, however it cannot activate apoptotic or necroptosis pathways. However, TNFR2 can function through the phosphatidylinositol 3-kinase (PI3K) pathway to promote cell survival and proliferation. This requires PI3K phosphorylation of the plasma membrane lipid phosphatidylinositol-4,5-bisphosphate (PIP2) resulting in the second messenger phosphatidylinositol 3,4,5-bisphosphate (PIP3). PKB/Akt then is recruited to the plasma membrane where PKB/Akt undergoes a conformational change and is phosphorylated by the Rictor/mammalian target of rapamycin (mTOR) complex.

Extrinsic pressures from the aged bone marrow (BM) microenvironment promotes CH expansion. For example, enhanced TNFα signaling in Dnmt3aR878H hematopoietic stem cells (HSCs) results in selective survival of Dnmt3a-mutant HSCs. Specific loss of TNFR1 results in depletion of Dnmt3a-mutant HSCs and their progeny, and that this is not replicated by loss of TNFR2. Therefore, targeting the TNF-TNFR1 signaling pathway reduces the survival advantage of Dnmt3a-mutant hematopoietic cells. Blocking TNFR1 pathway components therefore reduces CH and risk of leukemic transformation.

The current strategy most widely employed to block TNF signaling is using drugs like etanercept. This strategy is not effective in preventing CH because TNFR1 and TNFR2 have distinct and counteractive functions in CH. Specifically targeting TNFR1 offers the most effective strategy to prevent CH, which drives blood cancer risk and cardiovascular disease risk.

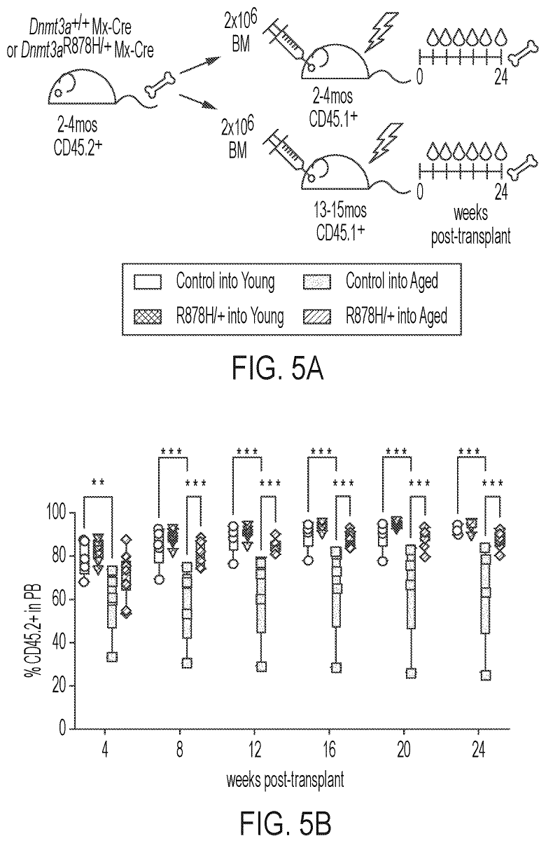

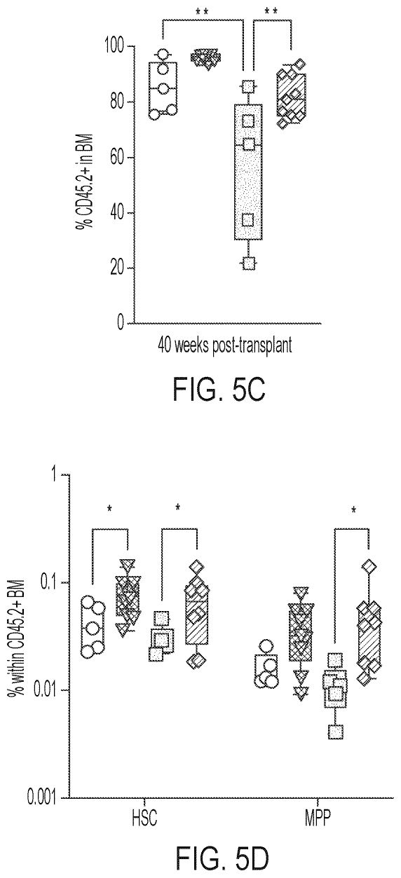

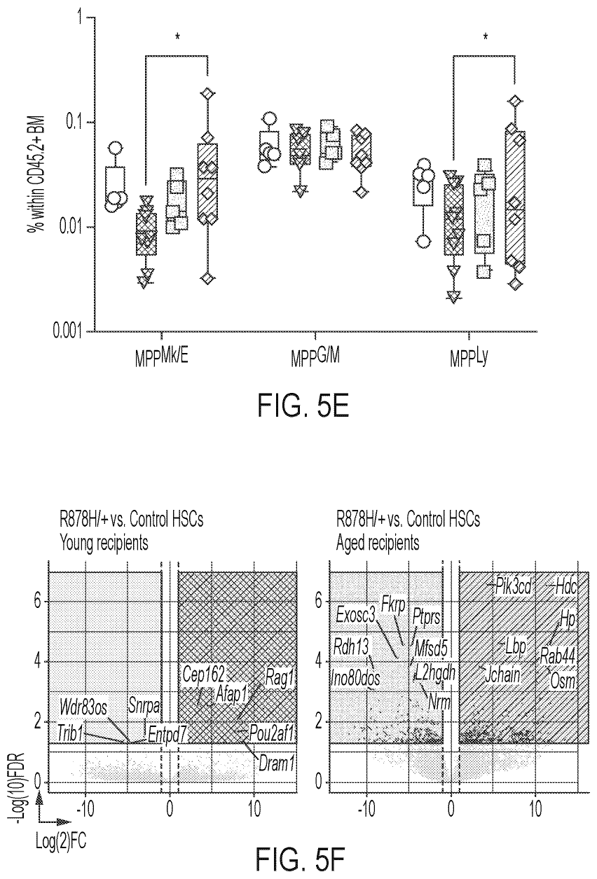

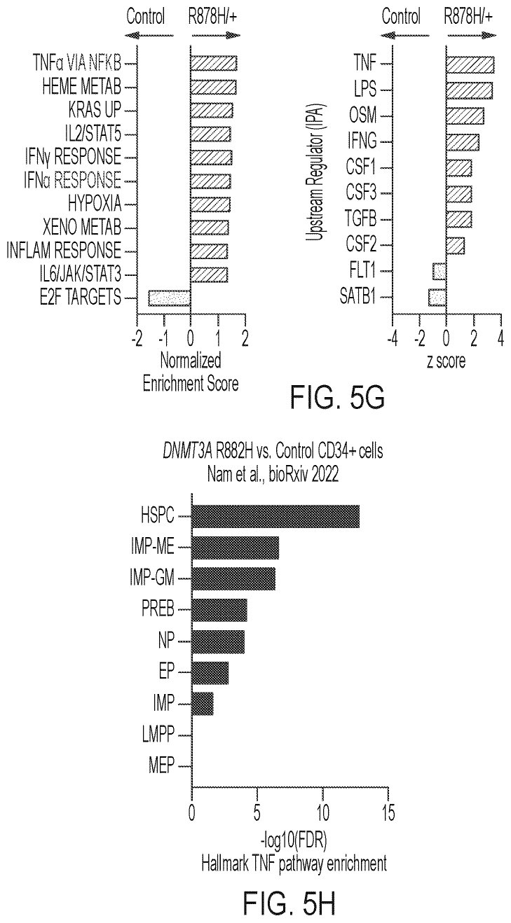

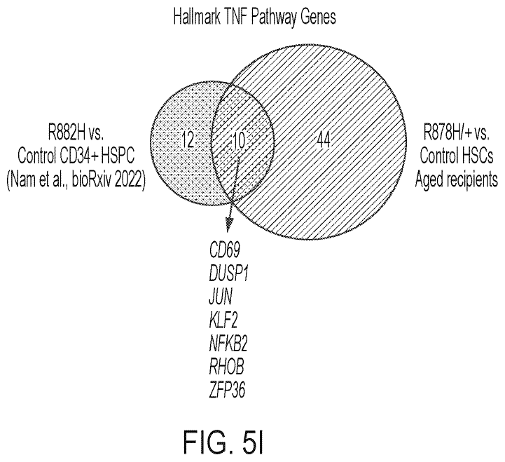

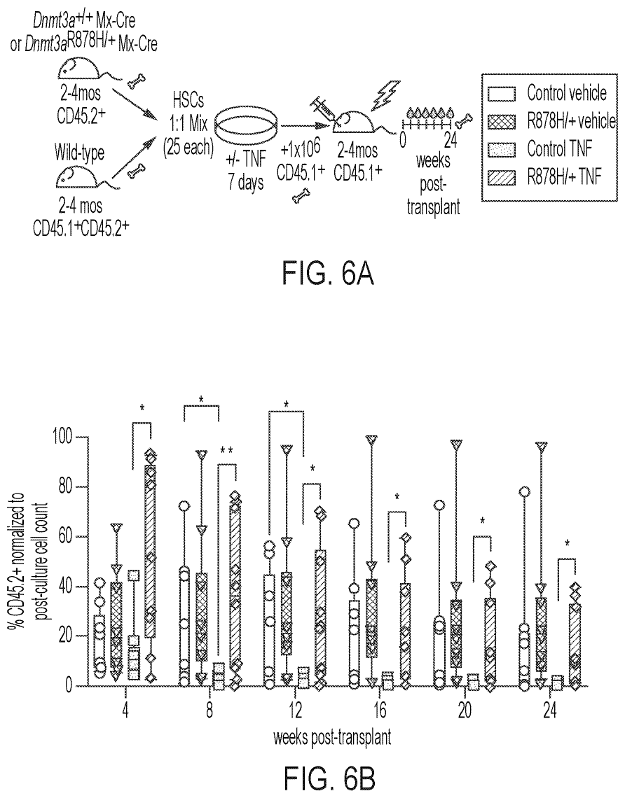

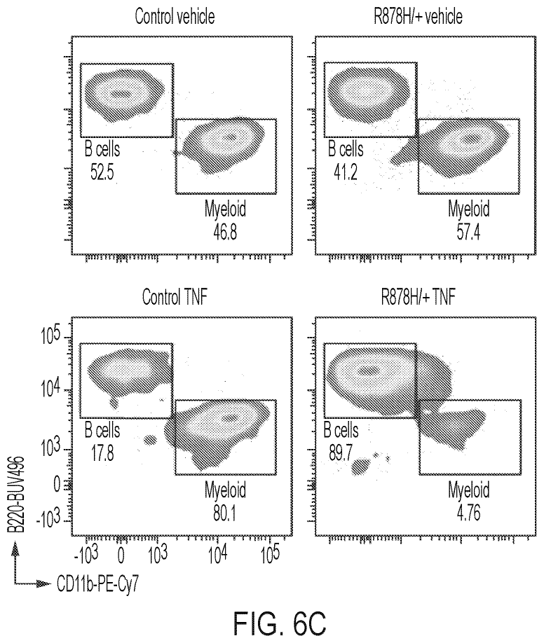

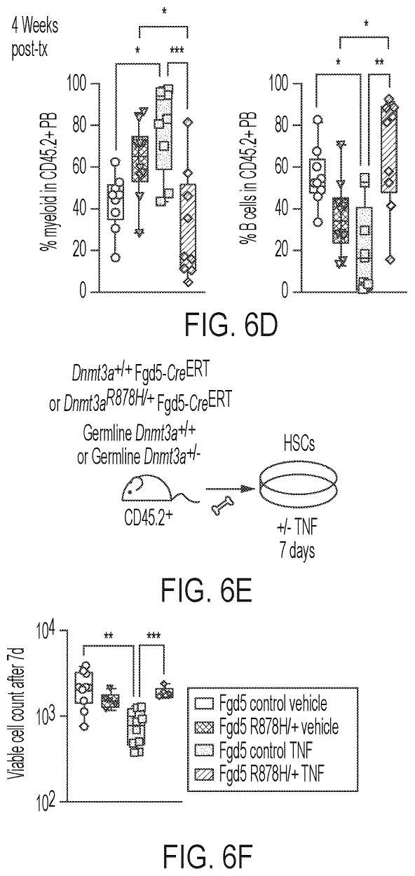

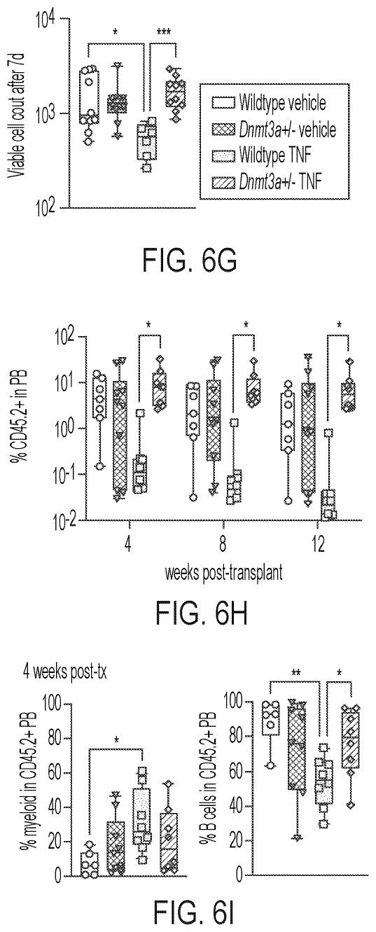

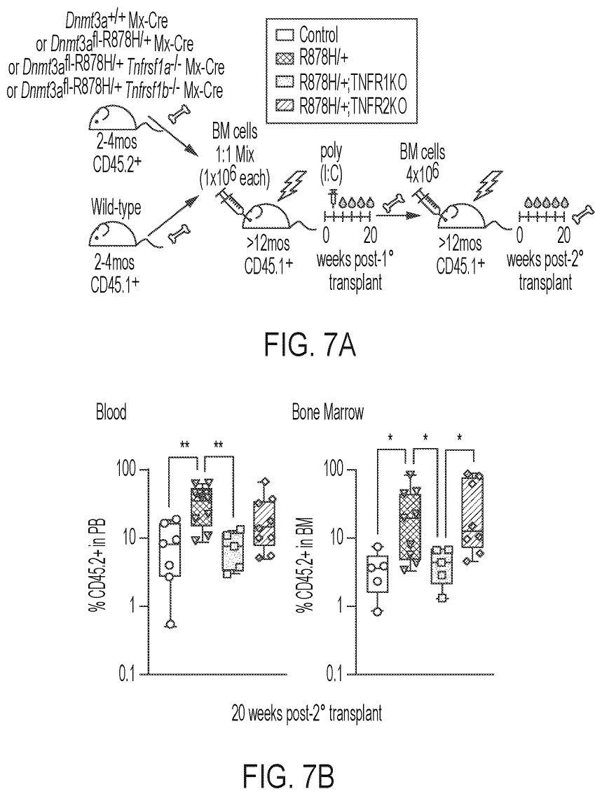

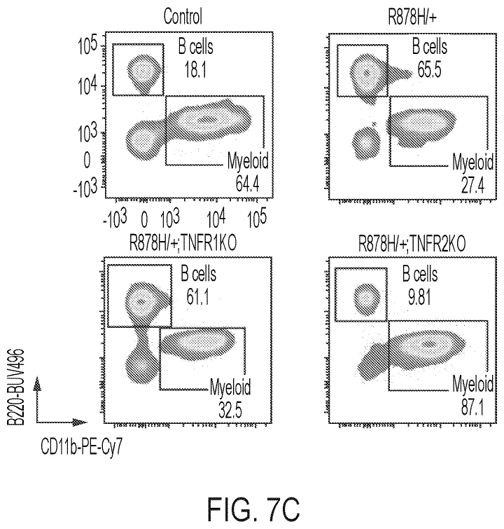

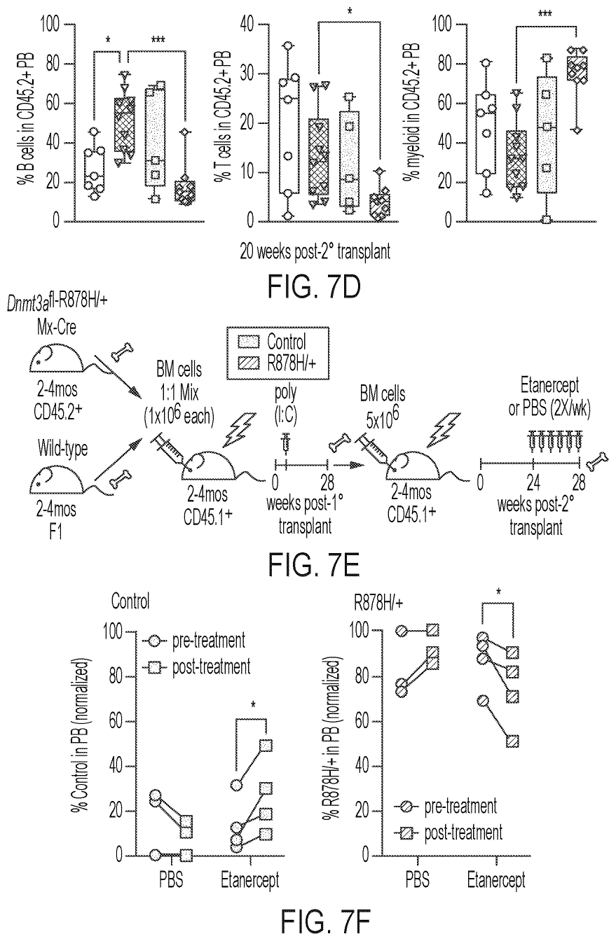

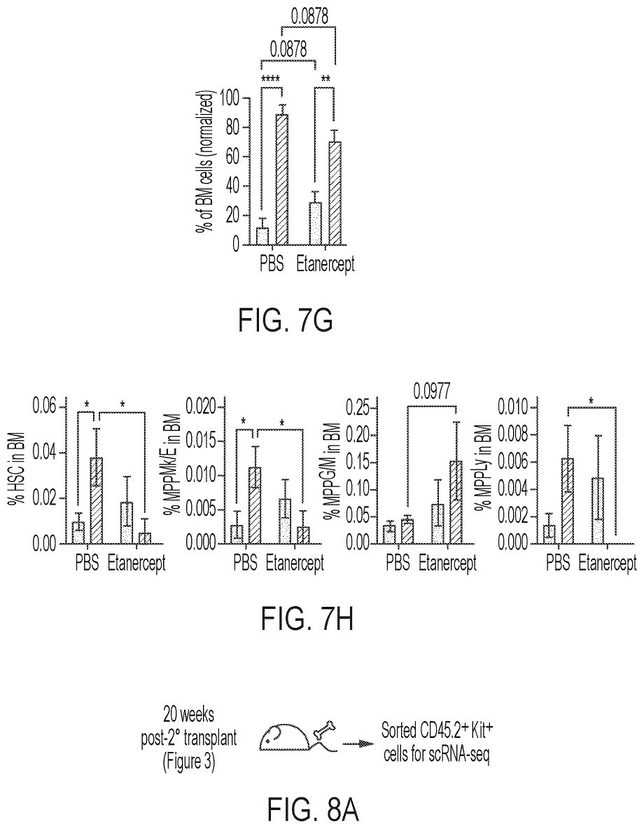

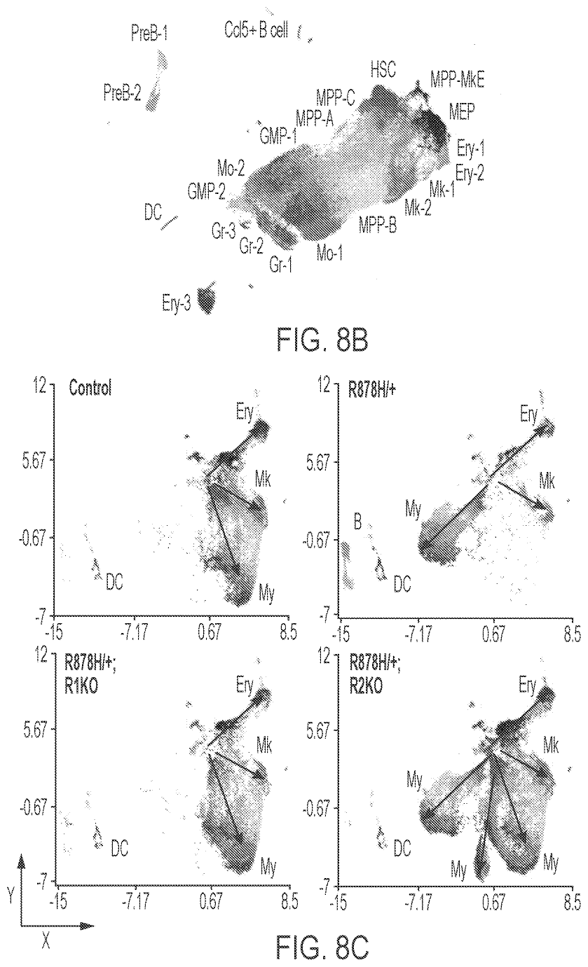

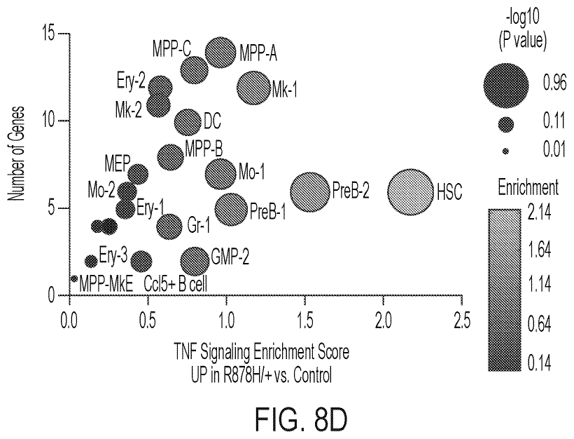

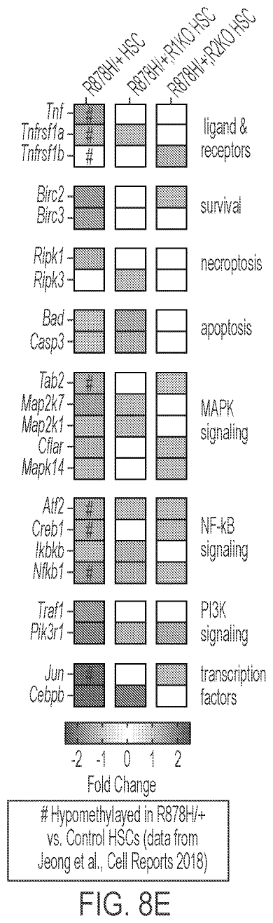

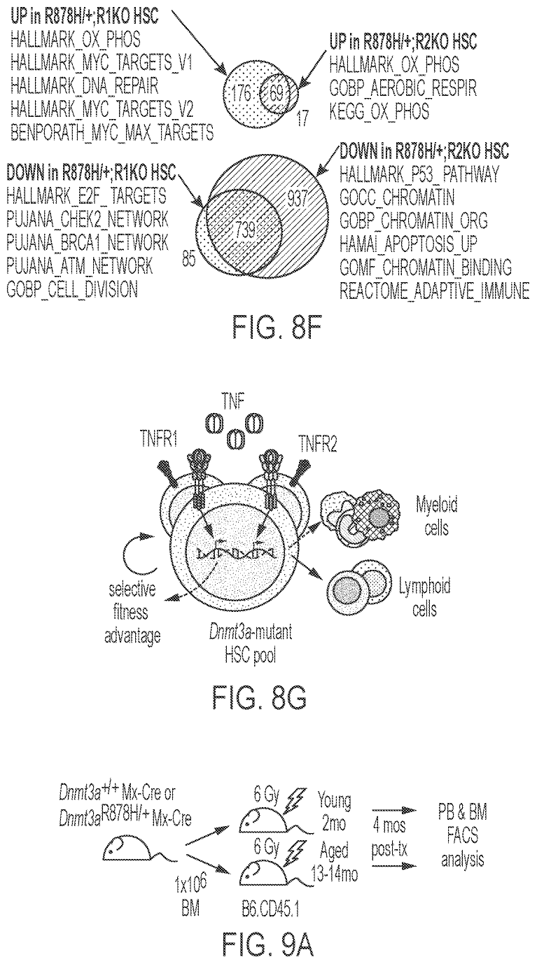

Using a mouse model of a common mutation in CH and acute myeloid leukemia (AML), DNMT3A^{R882H} (mouse Dnmt3a^{R878H}), the present disclosure provides data showing that transplant of Dnmt3a^{R878H} cells into aged recipients leads to accelerated clonal expansion compared to young recipients (FIGS. 5A-5C). This result indicates extrinsic pressures from the aged bone marrow (BM) microenvironment may promote CH expansion. Using transcriptomics and protein pathway analysis, enhanced TNFα signaling was identified in Dnmt3a^{R878H} hematopoietic stem cells (HSCs) reisolated from the aged BM (FIGS. 6A-6E). Adding recombinant TNFα to mixed cultures of wild-type and Dnmt3a^{R878H} HSCs resulted in preferential maintenance of Dnmt3a^{R878H} HSC progeny and depletion of wild-type progeny, supporting that elevated TNFα favors selective survival of Dnmt3a-mutant HSCs (FIGS. 7A-7C). To determine whether this phenotype was dependent upon TNF receptor 1 (TNFR1) versus TNF receptor 2 (TNFR2) signaling, Dnmt3a^{R878H} mice were crossed with targeted knockouts of either Tnfrsf1 (TNFR1) or Tnfrsf2 (TNFR2). Specific loss of TNFR1 results in depletion of Dnmt3a-mutant HSCs and their progeny, and this is not replicated by loss of TNFR2 (FIGS. 8A-8D). This work demonstrates that targeting the TNF-TNFR1 signaling axis is effective in reducing the survival advantage of Dnmt3a-mutant hematopoietic cells and suggests that targeted TNFR1 blocking antibodies and/or targeted small molecule inhibitors of downstream signaling factors in this pathway are promising strategies to reduce CH and risk of leukemic transformation.

In some embodiments, a method of treating clonal hematopoiesis includes administering to a subject a TNF signaling inhibitor in an amount effective to suppress clonal hematopoiesis. In some embodiments, a TNF signaling inhibitor is administered to a subject in an amount effective to suppress clonal hematopoiesis, relative to an untreated control hematopoiesis. In some embodiments, clonal hematopoiesis is suppressed by about 15-100 (15-100, 15-75, 15-50, 15-25, 25-100, 25-50, 25-75, 50-100, 50-75, or 75-100)% relative to an untreated control hematopoiesis. In some embodiments, clonal hematopoiesis is suppressed by about 15%, about 25%, about 50%, about 75%, about 100% relative to an untreated control hematopoiesis.

A “TNF signaling inhibitor” is an agent that disrupts the function of TNF such that signaling through downstream pathways is reduced or abolished. Examples of TNF signaling inhibitors include biologics, such as infliximab, etanercept, adalimumab, golimumab and certolizumab pegol, and small molecule drugs, such as the compound SPD-304. In some embodiments, the TNF signaling inhibitor is enteracept (Enbrel®).

In some embodiments, signs of elevated TNF signaling in a subject comprises increased RNA transcripts of TNF and TNFα signaling pathway components based on gene expression patterns. In some embodiments, RNA transcripts of TNF and TNFα signaling pathway components are elevated by about 15-100 (15-100, 15-75, 15-50, 15-25, 25-100, 25-50, 25-75, 50-100, 50-75, or 75-100)%. In some embodiments, RNA transcripts of TNF and TNFα signaling pathway components are elevated by about 15%, about 25%, about 50%, about 75%, about 100%.

In some embodiments, reducing TNF signaling through TNFR1 in a subject is measured by HSCs maintenance of self-renewal and engraftment ability.

In some embodiments, an antibody that specifically binds to TNFR1 comprises a blood protein produced in response to and counteracting a TNFR1 antigen and not a TNFR2 antigen.

### C. OSM/OSMR Signaling

In some embodiments, a method of treating clonal hematopoiesis includes modulating OSM signaling. Key components of hematopoietic stem cell (HSC) niches in the bone marrow (BM) includes endothelial and mesenchymal progenitor cells, adrenergic neurons, Schwann cells as well as HSC progenies such as megakaryocytes, macrophages and dendritic cells. The coordinated action of these niche cells and local physicochemical cues such as oxygenation levels enables the control of HSC behavior via the expression of an array of key regulators that control HSC quiescence and maintenance within BM niches. Some key regulators of HSC are induced in response to pro-inflammatory signaling. For example, the pro-inflammatory cytokine oncostatin M (OSM), a member of the IL-6 cytokine family has a role in regulating HSCs.

OSM influences numerous homeostatic and pathological processes depending on the tissue type and physiological context. OSM is produced by BM osteoblasts and macrophages and primarily expressed by hematopoietic cell types. The general response to OSM is to produce inflammatory signaling molecules and expression of factors that alter the extracellular matrix, cell proliferation and differentiation. Aberrant OSM expression promotes pathology and organ dysfunction. OSM has a four-helix bundle topology and engages receptor complexes composed of gp130 and a ligand specific receptor subunit. This signaling complex signals through LIFR or OSMR receptor proteins. Important residues of OSM that regulate interaction with LIFR or OSMR include F160 and K163. Residues that mediate OSM interaction with gp130 include Q16, Q20, G120 and N124. Like other cytokine receptors, the intracellular domain of gp130 and OSMR does not contain intrinsic kinase activity and require the receptor-associated Janus kinases (JAKs) to transduce signals. OSM signaling is propagated by both JAK1 and JAK2. JAK1 and JAK2 phosphorylate tyrosine residues in the cytoplasmic domains of gp130 and OSMR. Downstream of JAK1 and JAK2 activation is the mitogen-activated protein kinase (MAPK) cascade, the phosphatidylinositol-3-kinase (PI3K) cascade and the signal transducer and activator of transcription-3 (STAT3) cascade. Upon phosphorylation, STAT3 can enter the nucleus and alter gene expression. Through these pathways OSM promotes expression of a key regulators of HSC such as, E-selectin. E-selectin activates HSCs from quiescence to proliferation and regulates HSC self-renewal in vascular niches.

Transplantation of Dnmt3a-mutant HSCs into an aged bone marrow microenvironment accelerates their expansion and selective advantage. This selective advantage is mediated by an increase in signaling through the pro-inflammatory cytokine molecule Oncostatin M (OSM). Bone marrow fluid of aging mice contain elevated OSM levels resulting in greater downstream Stat3 phosphorylation compared to control HSCs. Depletion of the OSM receptor (Osmr-KO) in Dnmt3a-mutants results in HSCs that preferentially undergo differentiation to myeloid lineage cells. Therefore, elevated OSM signaling in the context of the aging bone marrow microenvironment contributes to positive selection of Dnmt3a-mutant HSCs. OSM/OSMR signaling, mediated through STAT3, is a mechanism by which CH-mutant stem cells expand in aging. Blocking or targeting OSM/OSMR reduces the selective advantage of clones and reduce risk of blood cancer.

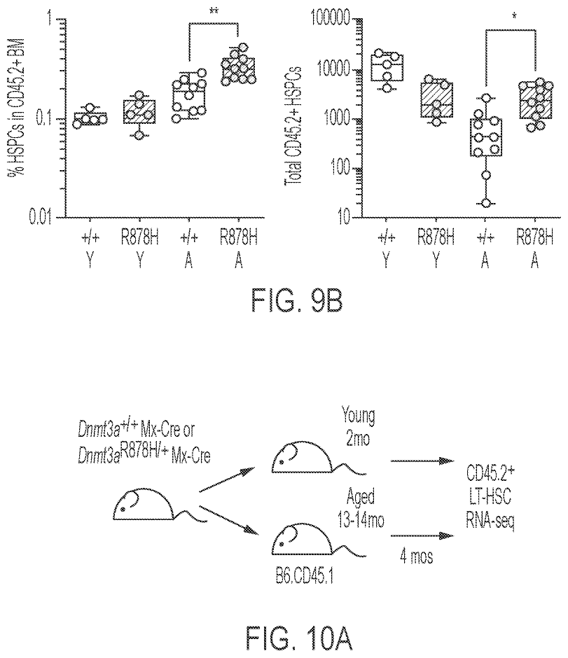

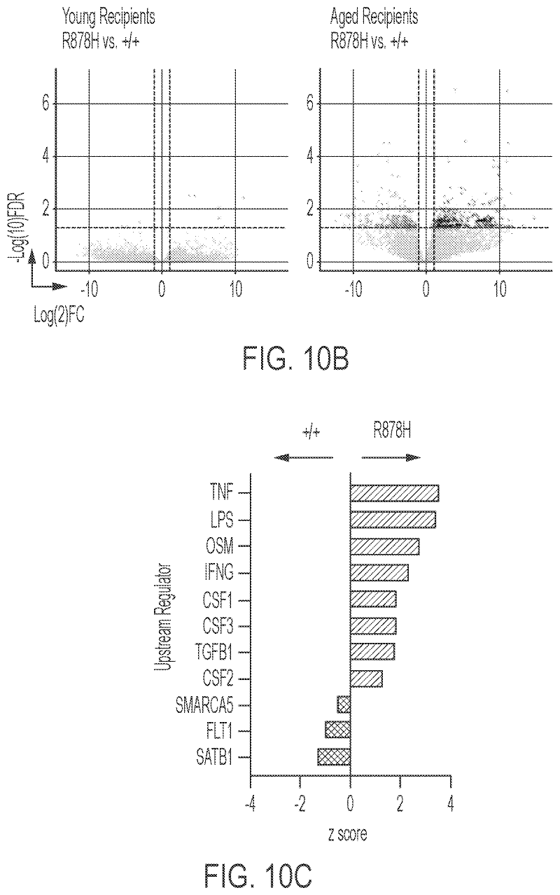

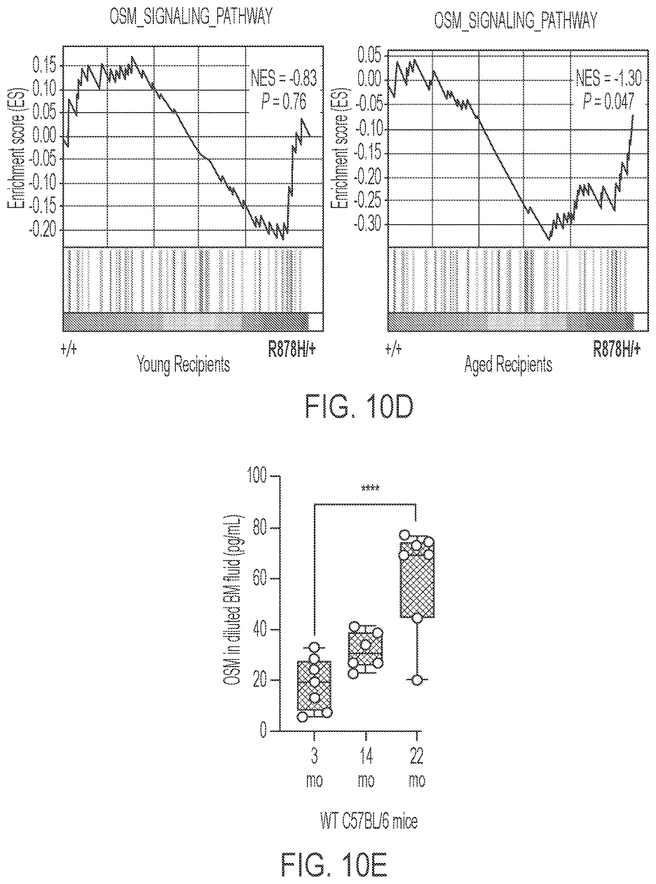

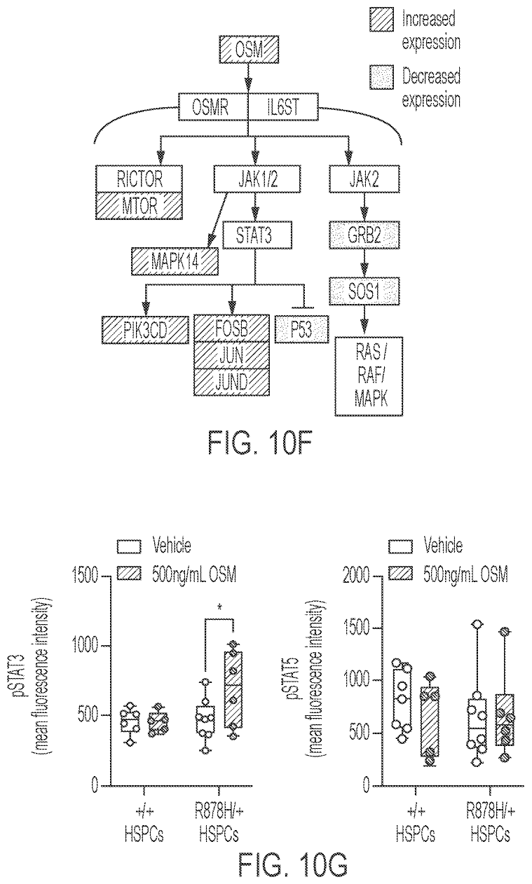

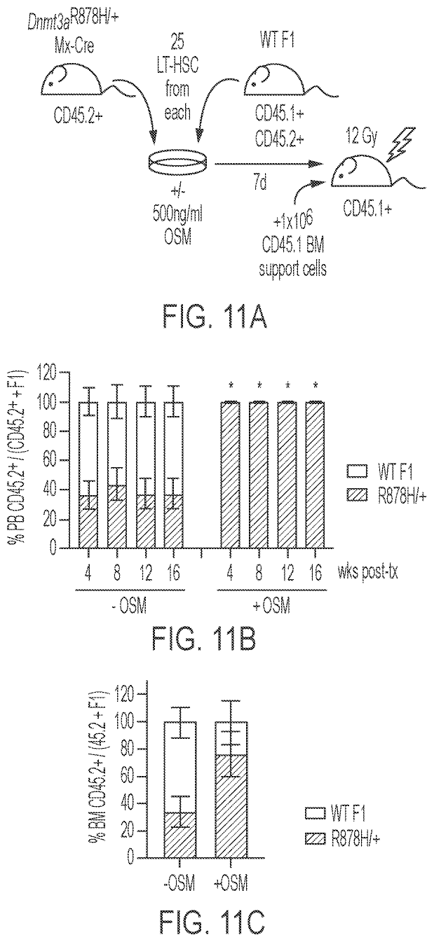

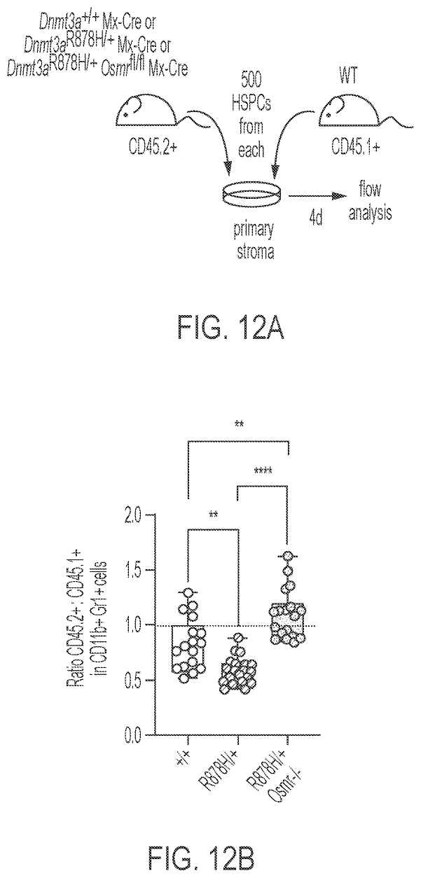

Using a mouse model with inducible expression of the murine equivalent of the most common mutation in human CH (DNMT3A-R882H; murine Dnmt3a-R878H), experiments showed that transplantation of Dnmt3a-mutant HSCs into an aged bone marrow microenvironment accelerates their expansion and selective advantage (FIGS. 9A-9B). This phenotype was correlated with a predicted increase in signaling through the pro-inflammatory cytokine molecule Oncostatin M (OSM) in Dnmt3a-mutant HSCs (FIGS. 10A-10D). In addition, OSM levels are elevated in bone marrow fluid of aging mice (FIG. 10E). Directly testing the role of OSM, it was found that stimulation of Dnmt3a-mutant HSCs with OSM resulted in greater downstream Stat3 phosphorylation compared to control HSCs, indicating differential signaling response (FIGS. 10F-10G). OSM-treated Dnmt3a-mutant HSCs had greater multi-lineage engraftment capacity in recipient mice compared to OSM-treated wild-type HSCs, supporting that this signaling molecule confers a selective advantage to Dnmt3a-mutant HSCs (FIGS. 11A-11C). When Dnmt3a mutant mouse are crossed with a knockout model of the OSM receptor (Osmr-KO), Dnmt3a-mutant Osmr-KO HSCs preferentially undergo differentiation to myeloid lineage cells (FIGS. 12A-12B). Together, this work suggests that elevated OSM signaling in the context of the aging bone marrow microenvironment contributes to positive selection of Dnmt3a-mutant HSCs. Thus, OSM/OSMR signaling is a compelling candidate for therapeutic targeting to reduce the selective advantage of mutant HSCs in the context of CH and decrease risk of CH-associated diseases such as blood cancer and coronary heart disease.

An “OSM signaling inhibitor” is an agent that disrupts the function of OSM such that signaling through downstream pathways is reduced or abolished. Examples of OSM signaling inhibitors include antibodies, biologics and small molecule drugs.

Some aspects of the present disclosure relate to methods of administration of an OSM signaling inhibitor that reduces OSM signaling in the subject. In some embodiments, OSM signaling is reduced by about 15-100 (15-100, 15-75, 15-50, 15-25, 25-100, 25-50, 25-75, 50-100, 50-75, or 75-100) %. In some embodiments, OSM signaling is reduced by about 15%, about 25%, about 50%, about 75%, about 100%.

In some embodiments, signs of elevated OSM signaling in a subject comprises increased RNA expression of OSM and OSM signaling pathway components based on gene expression patterns. In some embodiments, RNA expression of OSM and OSM signaling pathway component are elevated by about 15-100 (e.g., 15-100, 15-75, 15-50, 15-25, 25-100, 25-50, 25-75, 50-100, 50-75, or 75-100)%. In some embodiments, RNA expression of OSM and OSM signaling pathway components are elevated by about 15%, about 25%, about 50%, about 75%, about 100% relative to an untreated control hematopoiesis.

Activation of OSM signaling can also be measured by increases in phosphorylation of STAT3. In some embodiments, phosphorylation of STAT3 is increased by about 15-100 (e.g., 15-100, 15-75, 15-50, 15-25, 25-100, 25-50, 25-75, 50-100, 50-75, or 75-100)%. In some embodiments, phosphorylation of STAT3 is increased by about by about 15%, about 25%, about 50%, about 75%, about 100%.

Signs of elevated OSM signaling can also be measured based on HSC self-renewal and expansion from assays such as engraftment capacity of transplanted HSC. In some embodiments, engraftment capacity of transplanted HSC is increased by about 15-100 (e.g., 15-100, 15-75, 15-50, 15-25, 25-100, 25-50, 25-75, 50-100, 50-75, or 75-100)%. In some embodiments, engraftment capacity of transplanted HSC is increased by about by about 15%, about 25%, about 50%, about 75%, about 100.

HSC with elevated OSM signaling results in increased growth of Dnmt3a-mutant HSCs over wild-type cells in the peripheral blood and bone marrow of recipient.

In some embodiments, OSM signaling is elevated by about 15-100 (e.g., 15-100, 15-75, 15-50, 15-25, 25-100, 25-50, 25-75, 50-100, 50-75, or 75-100)%. In some embodiments, OSM signaling is elevated by about 15%, about 25%, about 50%, about 75%, about 100% relative to an untreated control hematopoiesis.

In some embodiments, signs of reduced OSM signaling in a subject comprises decreased RNA expression of OSM and OSM signaling pathway components based on gene expression patterns. Reduction of OSM signaling can also be measured by decreases or absence of STAT3 phosphorylation. Signs of reduced OSM signaling can also be measured based on HSC self-renewal and expansion from assays such as engraftment capacity of transplanted HSC. HSC with reduced OSM signaling results in similar or equal growth of Dnmt3a-mutant HSCs over wild-type cells in the peripheral blood and bone marrow of recipient.

In some embodiments, an antibody that specifically binds to OSM or OSMR comprises a blood protein produced in response to and counteracting an OSM or OSMR antigen and no other antigens.

Some aspects of the present disclosure relate to a method of treating clonal hematopoiesis in a subject in need thereof, by administering to the subject an OSM signaling inhibitor in an amount effective to suppress clonal hematopoiesis in the subject, relative to an untreated control. In some embodiments, an OSM signaling inhibitor is administered to a subject in an amount effective to suppress clonal hematopoiesis, relative to an untreated control hematopoiesis. In some embodiments, clonal hematopoiesis is suppressed by about 15-100 (e.g., 15-100, 15-75, 15-50, 15-25, 25-100, 25-50, 25-75, 50-100, 50-75, or 75-100)% relative to an untreated control hematopoiesis. In some embodiments, clonal hematopoiesis is suppressed by about 15%, about 25%, about 50%, about 75%, about 100% relative to an untreated control hematopoiesis.

### D. Antibodies

Some aspects of the present disclosure provide an antibody that binds specifically to TNFR1 in a subject. In some embodiments, the antibody that binds specifically to TNFR1 comprises a heavy chain. In some embodiments, the antibody that binds specifically to TNFR1 further comprises a light chain.

Some aspects of the present disclosure provide an antibody that binds specifically to OSM/OSMR in a subject. In some embodiments, the antibody that binds specifically to OSM/OSMR comprises a heavy chain. In some embodiments, the antibody that binds specifically to OSM/OSMR further comprises a light chain.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the methods described herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Any of the antibodies described herein can be either monoclonal or polyclonal. A “monoclonal antibody” refers to a homogenous antibody population and a “polyclonal antibody” refers to a heterogenous antibody population. These two terms do not limit the source of an antibody or the manner in which it is made.

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In other embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen, such as (mouse or human) OSM/OSMR. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a TNFR1 or OSM/OSMR epitope is an antibody that binds this TNFR1 or OSM/OSMR epitope, respectively, with greater affinity, avidity, more readily, and/or with greater duration than it binds to other TNFR1 or OSM/OSMR epitopes or non-TNFR1 or non-OSM/OSMR epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding. In some embodiments, the equilibrium dissociation constant (KD) between the antibody and NGly-1 is 100 pM to 1 μM. In some embodiments, the KD between the antibody and NGly-1 is 1 nM to 100 nM.

### Heavy Chain

A heavy chain is the large polypeptide subunit of an antibody. Heavy chains differ in size and composition, but are typically between 450 and 550 amino acids in length and are composed of a constant domain (HC Constant), comprising three or four immunoglobulin domains, and a variable domain (HC Variable), comprising a single immunoglobulin domain. The variable domain of the heavy chain is important for binding antigen. An immunoglobulin domain is a structure formed by the three-dimensional arrangement of beta-strands into parallel beta-sheets. There are five types of heavy chains in mammals which define the class of antibody, wherein IgA antibodies contain alpha (α) heavy chains, IgD antibodies contain delta (δ) heavy chains, IgE antibodies contain epsilon (ε) heavy chains, IgM antibodies contain mu (μ) heavy chains, and IgG antibodies contain gamma (γ) heavy chains.

### Light Chain

A light chain is the small polypeptide of an antibody. Light chains differ in size, but are typically between 210 and 217 amino acids in length and are composed of a constant domain (LC Constant), comprising a single immunoglobulin domain, and a variable domain (LC Variable), comprising a single immunoglobulin domain. There are two types of light chains in mammals, which are defined by the sequence of the constant region and classified as either kappa (κ) or lambda (λ). The variable domain of the light chain is important for binding antigen. Only one type of light chain is typically present in an antibody, so the two light chains within a single antibody are identical.

### Variable Region

Each antibody has a unique variable region composed of the variable domains of both heavy and light chains which contains the antigen binding site. The variable region is further subdivided into complementarity determining regions (CDRs) and framework (FR) regions. There are two variable domains on each antibody which typically, but not always, bind the same antigen.

### CDR

A complementarity-determining region (CDR) (also known as a hypervariable region, or HV) within the variable region has a high ratio of different amino acids in a given position, relative to the most common amino acid in that position. Three CDRs (CDR1, CDR2, CDR3) exist within heavy and light chains, which form flexible loops that directly contact a portion of the antigen's surface.

### Framework Region

The framework region (FR) within a variable region is composed of conserved amino acid sequences which separate CDR sequences. The FR regions form a beta-sheet structure which serves as a scaffold to hold the CDRs in position to contact the antigen surface. Four FR regions exist within each heavy and light chain.

### Constant Region

The constant region of an antibody is recognized by receptors on immune cells and proteins to initiate and regulate host defense mechanisms. The constant region of heavy chain polypeptides is identical in all antibodies of the immunoglobulin class, but differs between immunoglobulin classes. Heavy chains in Igγ, Igα, and Igδ contain a constant region composed of three immunoglobulin domains and a hinge region for increased flexibility. Heavy chains in Igμ and Igε contain a constant region composed of four immunoglobulin domains. The constant region of light chain polypeptides is composed of a single immunoglobulin domain.

### II. Blood Cancer

Blood cancers, also referred to as hematologic cancers, start in the bone marrow, which is where blood cells are produced. Normally functioning blood cells fight off infections and produce new blood cells. Blood cancers occur when abnormal blood cells grow out of control and interrupt the function of normal blood cells. There are three main types of blood cancer, leukemia, lymphoma and myeloma. Leukemia originates in the blood and bone marrow and occurs when the body creates too many abnormal white blood cells. When leukemia occurs, the bone marrow's ability to produce red blood cells and platelets are diminished. Non-Hodgkin and Hodgkin lymphomas are blood cancers that develop in the lymphatic system from white blood cells called lymphocytes. Hodgkin lymphoma is characterized by the presence of an abnormal lymphocyte called the Reed-Sternberg cell. Myeloma is a blood cancer of the blood's plasma cells which, is a type of white blood cell made in the bone marrow. Symptoms of these blood cancers include fever and frequent infections, fatigue, nausea, unexplained weight loss, bone/joint pain, headaches, shortness of breath and swollen lymph nodes. Blood cancers account for approximately 10% of cancer diagnoses with over 900,000 people worldwide diagnosed with blood cancer every year. In the United States, 68,000 people die from blood cancers every year. People with CH have an increased risk of developing blood cancers, specifically myelodysplastic syndrome (also called preleukemia) and acute myeloid leukemia.

In some embodiments, subject at risk of blood cancer comprises a subject exhibiting a sign of CH. This includes subjects with age-related mutations associated with clonal hematopoietic expansion and malignancies, mainly DNMT3A, TET2, and ASXL1.

### III. Cardiovascular Disease

Cardiovascular diseases, a group of disorders relating to the heart and blood vessels, are the leading cause of death worldwide. Cardiovascular diseases include coronary heart disease, which is atherosclerosis in the heart that results in the narrowing of arteries carrying blood to the heart. Globally, 17.9 million people die from cardiovascular disease worldwide and in the United States approximately 650,000 deaths are due to cardiovascular disease. There are approximately 18 million U.S. citizens over the age of 20 with heart disease. Patients with cardiovascular disease often do not show any symptoms and a heart attack or stroke may be the first indication of disease. Other possible symptoms of heart disease include shortness of breath, fatigue, irregular heartbeats, and chest pain. Incidence of cardiovascular disease increases with age. Most frequently, age-related cardiovascular disease is marked by expansion of hematopoietic clones with loss-of-function mutations in the genes DNMT3A, TET2, and ASXL1.1-3. The presence of CH mutations in patient blood cells are associate with a doubling of the risk of coronary heart disease and an increased risk for death from any cause including, coronary heart disease.

In some embodiments, subject at risk of cardiovascular disease comprises a subject exhibiting a sign of CH. This includes subjects with age-related mutations associated with clonal hematopoietic expansion and malignancies, mainly DNMT3A, TET2, and ASXL1.

As described herein, a “subject in need thereof” refers to a subject in need of treatment for clonal hematopoiesis or a disorder arising from clonal hematopoiesis (e.g., blood cancer or cardiovascular disease). As described herein, a “subject” refers to mammal. The mammal may be selected from, but is not limited, a human, primate, rat, mouse, dog, cat, cow, goat, camel, sheep, or pig. The terms “subject in need thereof” and “subject” may be used interchangeably herein.

In some embodiments, the subject exhibits signs of a sign of clonal hematopoiesis. In some embodiments, the subject exhibits signs of defects in mitochondrial metabolism hematopoiesis. In some embodiments, the subject exhibits signs of elevated TNF signaling. In some embodiments, the subject exhibits signs of elevated OSM signaling. In some embodiments, the subject is at risk of blood cancer. In some embodiments, the subject is at risk of cardiovascular disease. In some embodiments, the subject is a human who exhibits signs of a sign of clonal hematopoiesis. In some embodiments, the subject is a human who exhibits signs of defects in mitochondrial metabolism hematopoiesis. In some embodiments, the subject is a human who exhibits signs of elevated TNF signaling. In some embodiments, the subject is a human who exhibits signs of elevated OSM signaling. In some embodiments, the subject is a human who is at risk of blood cancer. In some embodiments, the subject is a human who is at risk of cardiovascular disease.

In some embodiments, the subject may be a newborn, an infant, a toddler, a child or an adult. In some embodiments, the subject may be a human subject that is a newborn, an infant, a child, or an adult. In some embodiments, the adult is at least 18-100 (e.g., 18-100, 18-90, 18-80, 18-70, 18-65, 18-60, 18-50, 18-40, 18-30, 18-20, 20-100, 20-90, 20-80, 20-70, 20-65, 20-60, 20-50, 20-40, 20-30, 30-100, 30-90, 30-80, 30-70, 30-65, 30-60, 30-50, 30-40, 40-100, 40-90, 40-80, 40-70, 40-65, 40-60, 40-50, 50-100, 50-90, 50-80, 50-70, 50-65, 50-60, 60-100, 60-90, 60-80, 60-70, 60-65, 65-100, 65-90, 65-80, 65-70, 70-100, 70-90, 70-80, 80-100, 80-90, 90-100) years of age. In some embodiments, the adult is at least 18, at least 20, at least 30, at least 40, at least 50, at least 60, at least 65, at least 70, at least 80, at least 90, at least 100 years old.

Some aspects of the present disclosure describe method of administering a therapeutic agent (e.g., mitochondria-targeted antioxidant, a TNF signaling inhibitor, an OSM signaling inhibitor) to the subject. In some embodiments, the therapeutic agents may be formulated in compositions for administration to the subject. The compositions may be selected from, but not limited to, liquids, aerosols, solutions, inhalants, mists, sprays, solids, powders, ointments, pastes, creams, lotions, gels, or patches. The compositions may be administered using a desirable route, including, but not limited to, pulmonary, inhalation, intranasal, oral, buccal, sublingual, parenteral, subcutaneous, intravenous, intramuscular, intraperitoneal, intrapleural, intrathecal, transdermal, transmucosal, or rectal.

### EXAMPLES

Clonal hematopoiesis resulting from enhanced fitness of mutant hematopoietic stem cells (HSCs) associates with both favorable and unfavorable health outcomes related to the types of mature mutant blood cells produced, but how this lineage output is regulated is unclear. Using a mouse model of a clonal hematopoiesis-associated mutation, DNMT3A^{R882/+} (Dnmt3a^{R878H/+}), aging-induced TNFα signaling was found to promote the selective advantage of mutant HSCs and stimulate production of mutant B lymphoid cells. Genetic loss of TNFα receptor TNFR1 ablated the selective advantage of mutant HSCs without altering their lineage output, while loss of TNFR2 resulted in overproduction of mutant myeloid cells without altering HSC fitness. These results nominate TNFR1 as a target to reduce clonal hematopoiesis and risk of associated diseases, and support a model wherein clone size and mature blood lineage production can be independently controlled to modulate favorable and unfavorable CH outcomes.

### Example 1. Altered Mitochondrial Metabolism is a Targetable Dependency in Dnmt3a-Mutant Clonal Hematopoiesis

Recent work (Young et al., Cell Stem Cell 2021) demonstrated that decline in Insulin-Like Growth Factor 1 (IGF1) in the aging bone marrow microenvironment is a major factor causing impaired function and myeloid-biased hematopoiesis from hematopoietic stem cells (HSCs). Here, we evaluated the extent to which decline in IGF1 in the bone marrow microenvironment may promote or favor growth of Dnmt3a-mutant HSCs, leading to clonal hematopoiesis. First, we transplanted control or Dnmt3a-mutant hematopoietic cells into recipient mice that were wild-type or had a conditional knockout (cKO) of Igf1. We observed that transplant of Dnmt3a-mutant cells into Igf1 cKO recipient mice resulted in expansion of Dnmt3a-mutant HSCs in the bone marrow (FIG. 1). This result suggests that aging-associated decline in IGF1 in the bone marrow microenvironment will favor expansion of Dnmt3a-mutant over wild-type HSCs, promoting clonal hematopoiesis.

IGF1 signaling is well understood to result in activation of downstream signaling through Akt and mTOR in many cell types, including HSCs (Young et al., Cell Stem Cell). To test the extent to which inhibiting mTOR could replicate loss of IGF1 signaling in the context of the above experiment, control or Dnmt3a-mutant hematopoietic cells were transplanted into recipient mice that were then treated with the mTOR inhibitor rapamycin (eRAPA) by supplementation in their diet. Transplant of Dnmt3a-mutant cells into rapamycin-treated recipient mice resulted in expansion of Dnmt3a-mutant HSCs in the bone marrow (FIG. 2). This result supports that aging-associated decline in IGF1-Akt-mTOR signaling in the bone marrow microenvironment will favor expansion of Dnmt3a-mutant over wild-type HSCs, promoting clonal hematopoiesis.

Previous work (Young et al., Cell Stem Cell 2021) demonstrated that loss of bone marrow microenvironment-derived IGF1 impaired mitochondrial metabolism and morphology in wild-type HSCs, and that recombinant IGF1 restored mitochondrial morphology in aged HSCs. To evaluate the extent to which targeting defects in mitochondrial metabolism might improve wild-type HSC function and thus reduce the selective advantage of Dnmt3a-mutant HSCs, cells were treated with the mitochondrially targeted antioxidant mitoquinol (MitoQ) in vitro and in vivo. In in vitro experiments testing myeloid differentiation of hematopoietic stem and progenitor cells, supplementation with MitoQ did not impact differentiation of wild-type control cells but did reduce number of progeny produced from Dnmt3a-mutant cells (FIG. 3). In vivo treatment with MitoQ after transplant of wild-type or Dnmt3a-mutant cells into aged recipient mice resulted in reduced contribution of Dnmt3a-mutant cells to hematopoiesis (FIG. 4). Together, this data suggests that treatment with MitoQ negatively and specifically impacts function of Dnmt3a-mutant HSCs such that it reduces their selective advantage in competition with wild-type HSCs. Treatment with MitoQ or other methods to improve mitochondrial metabolism are attractive targets to consider in the treatment of clonal hematopoiesis and prevention of leukemia.

Clonal hematopoiesis resulting from enhanced fitness of mutant hematopoietic stem cells (HSCs) associates with both favorable and unfavorable health outcomes related to the types of mature mutant blood cells produced, but how this lineage output is regulated is unclear. Using a mouse model of a clonal hematopoiesis-associated mutation, DNMT3A^{R882/+} (Dnmt3a^{R878H/+}), aging-induced TNFα signaling was found to promote the selective advantage of mutant HSCs and stimulate production of mutant B lymphoid cells. Genetic loss of TNFα receptor TNFR1 ablated the selective advantage of mutant HSCs without altering their lineage output, while loss of TNFR2 resulted in overproduction of mutant myeloid cells without altering HSC fitness. These results nominate TNFR1 as a target to reduce clonal hematopoiesis and risk of associated diseases, and support a model wherein clone size and mature blood lineage production can be independently controlled to modulate favorable and unfavorable CH outcomes.

### Example 2. Targeting the TNF-TNFR1 Signaling Axis Depletes Dnmt3a-Mutant Hematopoietic Stem Cells (HSCs) in Clonal Hematopoiesis

Our group recently found that the middle-aged bone marrow (BM) microenvironment drives HSC aging. This work established an experimental paradigm to evaluate potency of Dnmt3a^{R878H/+} HSCs in the aged BM microenvironment and identify HSC-extrinsic factors that modulate their selective advantage. We transplanted Dnmt3a^{R878H/+} HSCs into young and middle-aged recipient mice (FIG. 5A). Dnmt3a^{R878H/+} HSCs transplanted into aged recipients generated greater long-term multilineage hematopoiesis compared to control HSCs (FIGS. 5A-5C) and gave rise to expanded HSC and multipotent progenitor (MPP) populations (FIGS. 5D-5E). In addition, aged recipients had higher proportions of Dnmt3a^{R878H/+} megakaryocyte and erythroid-primed MPPs (MPP^{Mk/E}) and lymphoid-primed MPPs (MPP^{Ly}) (FIG. 5E). The latter was consistent with increased mature Dnmt3a^{R878H/+} B lymphoid cells (data not shown). No change in frequency of mature T lymphoid cells was observed, which may be explained in part by thymic involution during aging. Together, we find that Dnmt3a^{R878H/+} HSCs have enhanced selective advantage over wild-type HSCs as well as altered lineage output in the context of the aged BM microenvironment.

To identify molecular signatures underlying expanded Dnmt3a^{R878H/+} hematopoiesis in the aged BM microenvironment, we performed RNA-seq on independent biological replicates of HSCs re-isolated from young and aged recipient mice. Our experimental design specified only a sublethal dose of irradiation to recipient mice, to better preserve HSC-extrinsic signals from the BM microenvironment (data not shown). A greater number of differentially expressed genes in Dnmt3a^{R878H/+} vs. control HSCs were found in aged compared to young recipient mice (FIG. 5F). Using gene and pathway enrichment analyses, TNFα was identified as the top enriched gene signature and predicted upstream regulator in Dnmt3a^{R878H/+} HSCs in aged mice (FIG. 5G). Complementary to this, we found increased levels of TNFα in bone marrow fluid of aged compared to young mice, as well as mice transplanted with Dnmt3a^{R878H/+} vs. control hematopoietic cells (data not shown). This is consistent with a previous report that circulating TNFα is significantly increased in humans with DNMT3A-mutant CH, relative to other CH mutations. A previously described TNFα-induced transcriptional program in HSCs, enriched in pro-survival genes, was elicited in Dnmt3a^{R878H/+} HSCs in aged mice (data not shown). In addition, Dnmt3a^{R878H/+} HSCs in aged mice sustained expression of transcriptional programs that define mouse and human HSCs (data not shown). In humans, DNMT3A^{R882H/+} CD34⁺ HSPCs were enriched for TNFα pathway genes compared to DNMT3A^{+/+} CD34⁺ HSPCs isolated from the same individuals (FIG. 5H). Several TNFα target genes were commonly upregulated in human DNMT3A^{R882H/+} CD34⁺ HSPCs and mouse Dnmt3a^{R878H/+} HSCs, including JUN and NFKB2 (FIG. 5I). Thus, the selective advantage of mouse and human DNMT3A-mutant HSCs positively correlates with elevated TNFα signaling.

To assess the extent to which TNFα directly promotes young Dnmt3a-mutant HSC survival, we added recombinant TNFα to mixed cultures of wild-type and Dnmt3a-mutant HSCs in media that sustains HSC self-renewal (FIG. 6A). TNFα treatment reduced the number of control but not Dnmt3a^{R878H/+} cells produced over the culture period (data not shown). Post culture, cells were transplanted into recipient mice to assess HSC function. TNFα-treated control HSCs did not sustain long-term multilineage engraftment (FIG. 6B). In contrast, TNFα-treated Dnmt3a^{R878H/+} HSCs increased production of mature hematopoietic cells in the short term (4 weeks post-transplant) followed by sustained multilineage engraftment. In addition, TNFα stimulation transiently increased Dnmt3a^{R878H/+} B lymphoid cell production (FIGS. 6C-6D), in contrast to myeloid regeneration from TNFα-treated control HSCs as has been previously reported. In the bone marrow, we observed trends toward reduced HSC, MPP^{Mk/E}, and MPP^{G/M} populations from TNFα-treated control HSCs, which was not observed in TNFα-treated Dnmt3a^{R878H/+} HSCs (data not shown). Thus, TNFα-driven myeloid regeneration at the expense of HSC self-renewal is disrupted in Dnmt3a^{R878H/+} HSCs. Instead, TNFα-treated Dnmt3a^{R878H/+} HSCs favor lymphoid regeneration and maintain their self-renewal. To assess this in complementary models, we used tamoxifen-inducible Fgd5-Cre-driven Dnmt3a^{R878H/+} mice as well as germline Dnmt3a^{+/-} mice to isolate HSCs for TNFα stimulation (FIG. 6E). TNFα treatment reduced the number of control but not Fgd5-Cre Dnmt3a^{R878H/+} cells (FIG. 6F) or Dnmt3a^{+/-} cells (FIG. 6G) over the culture period. Upon transplant, TNFα-treated Dnmt3a^{+/-} HSCs sustained multilineage engraftment at a higher frequency than TNFα-treated control HSCs (FIG. 6H). In addition, TNFα stimulation transiently increased Dnmt3a^{+/-} B lymphoid relative to myeloid cell production (FIG. 6I). Thus, TNFα-induced HSC survival and disrupted myeloid regeneration are broadly relevant to Dnmt3a-mutant clonal hematopoiesis.

TNFα signaling occurs through two distinct TNFα receptors, TNFR1 (Tnfrsf1a) and TNFR2 (Tnfrsf1b). Both TNFR1 and TNFR2 are expressed on HSC and MPP populations and are not altered in surface expression between control and Dnmt3a^{R878H/+} mice (data not shown). To determine which of these receptors are responsible for TNFα-mediated selective advantage of Dnmt3a^{R878H/+} HSCs and B lymphoid cell production, we crossed Dnmt3a^{R878H/+} mice with Tnfrsf1a or Tnfrsf1b knockout mice (data not shown) and tested HSC function by competitive serial BM transplantation into aged recipients (FIG. 7A). Loss of TNFR1, but not TNFR2, eliminated the selective advantage of Dnmt3a^{R878H/+} PB and BM cells in secondary transplant recipients (FIG. 7B). No change in engraftment was observed in TNFR1 knockout-only controls (data not shown), demonstrating that this is a specific dependency of Dnmt3a^{R878H/+} cells. In contrast, loss of TNFR2, but not TNFR1, reduced the proportion of B and T lymphoid cells and increased the proportion of myeloid cells (FIGS. 7C-7D) without altering overall white blood cell production (data not shown). Myeloid-biased hematopoiesis was also observed in TNFR2 knockout-only controls but only late in the post-secondary-transplant period (data not shown) and it did not increase neutrophil count (data not shown). Thus, distinct TNFα receptors mediate Dnmt3a^{R878H/+} HSC regenerative capacity versus lineage output. We compared our TNFR knockout phenotypes to pharmacological pan-TNFα blockade using etanercept (FIG. 7E). Etanercept reduced the competitive PB advantage of Dnmt3a^{R878H/+} cells (FIG. 7F), trended toward reduction in BM engraftment (FIG. 7G), and reduced the frequency of Dnmt3a^{R878H/+} HSC, MPP^{Mk/E}, and MPP^{Ly} populations (FIG. 7H). In contrast, etanercept produced a trend toward increase of Dnmt3a^{R878H/+} myeloid-primed multipotent progenitors MPP^{G}/M. Thus, pan-TNF inhibition results in a mix of our observed TNFR knockout phenotypes, that is, reduced selective advantage of Dnmt3a^{R878H/+} hematopoiesis as well as myeloid lineage bias at the stem/progenitor cell level.

To interrogate mechanisms by which TNFα signaling through different receptors impacts Dnmt3a^{R878H+} cells, we harvested donor-derived hematopoietic stem and progenitor cells from secondary transplant recipient mice for single cell RNA-sequencing (n=3-4 biological replicates per genotype) (FIG. 8A). After quality control filtering (data not shown), a total of 64,830 cells clustered into 22 populations (FIG. 8B). These clusters were classified based on published data to identify HSC, multipotent progenitor, and lineage-specified progenitor subsets (data not shown). All clusters included cells expressing similar levels of Tnfrsf1a as well as Tnfrsf1b (data not shown). Pseudotime trajectory analysis revealed that myeloid progenitor differentiation from HSCs (My) followed a distinct path in Dnmt3a^{R878H/+} vs. control BM (FIG. 8C), whereas erythroid (Ery) and megakaryocyte (Mk) progenitor differentiation paths were similar. Loss of TNFR1 fully corrected the aberrant Dnmt3a^{R878H/+} HSC to myeloid progenitor differentiation trajectory to closely resemble control BM, while loss of TNFR2 created multiple myeloid differentiation trajectories from Dnmt3a^{R878H/+} HSCs. These data are highly consistent with our functional observations that loss of TNFR1 eliminates Dnmt3a^{R878H/+} selective advantage and loss of TNFR2 biases toward myeloid cell production. Within subsets of hematopoietic stem and progenitor cells, TNF signaling was most enriched in Dnmt3a^{R878H/+} vs. control HSCs (FIG. 8D), supporting that TNF-induced phenotypes are initiated at the HSC level. Indeed, many downstream TNF targets were increased in expression in Dnmt3a^{R878H/+} vs. control HSCs, and several of these target genes are known to be hypomethylated in Dnmt3a^{R878H/+} HSCs (FIG. 8E). Examining the proportion of single HSCs that express Tnfrsf1a and/or Tnfrsf1b transcripts, we found that the majority (˜70%) of HSCs had detectable Tnfrsf1a transcript only, ˜15% of HSCs had detectable Tnfrsf1b transcript only, and the remaining ˜15% of HSCs had both Tnfrsf1a and Tnfrsf1b transcripts (data not shown). This was modestly altered in Dnmt3a^{R878H/+} HSCs with a trend toward more single Tnfrsf1a-expressing HSCs. Knockout of the opposing receptor did not transcriptionally alter receptor expression (data not shown). Focusing on unique transcriptional changes in Dnmt3a^{R878H/+} TNFR1 vs. TNFR2 knockout HSCs, we found that loss of TNFR1 resulted in increased expression of mediators of apoptosis, initiation factors for DNA repair and checkpoint activation, increased expression of Cebpb, and decreased cell division (FIGS. 8E-8F). In contrast, loss of TNFR2 resulted in increased expression of the apoptosis inhibitor Birc2, decrease in tumor suppressor p53, dysregulation of chromatin organization and decreased B and T lymphoid ‘adaptive immune’ signatures. Thus, TNFα-TNFR1 signaling promotes Dnmt3a^{R878H/+} HSC competitive advantage through evasion of apoptosis, accumulation of DNA damage, self-renewal, and cell cycling. In contrast, TNFα-TNFR2 signaling promotes lymphoid cell production from Dnmt3a^{R878H/+} HSCs, and restrains myeloid cell production, through chromatin regulation and expression of lymphoid-specifying genes.

Inhibition of pro-inflammatory cytokines, including TNFα, has been proposed as a generalizable strategy to reduce fitness of CH-mutant HSCs and risk of CH-associated disease states that are related to abnormal production of pro-inflammatory myeloid cells. Our work suggests that while pan-TNF inhibition does reduce Dnmt3a^{R878H/+} HSC fitness, it also results in more complex and potentially detrimental effects due to unrestrained Dnmt3a^{R878H/+} myeloid cell production. This is consistent with increased risk of inflammation reported as a severe side effect of pan-TNF inhibitor treatment. Here, we have found that targeting TNFR1 versus TNFR2 can separate molecular programs dictating HSC fitness from myeloid cell production such that targeting TNFR1 specifically reduces Dnmt3a-mutant HSC fitness while maintaining lineage-balanced output. Furthermore, targeting TNFR1 in wild-type HSCs was not observed to have detrimental consequences on hematopoietic output over serial transplantation, supporting that TNFR1 is a unique therapeutic vulnerability of Dnmt3a-mutant clones. Given that we identified subsets of Dnmt3a-mutant HSCs expressing one or both TNF receptors, further study is needed to determine the extent to which these represent functionally distinct HSC populations.

Reported potential beneficial aspects of DNMT3A-mutant clonal hematopoiesis thus far have related to lymphoid cell production including increased anti-tumor T cells and maintenance of T cell immunity during aging. Here, we find that TNFα-driven lymphoid cell production from Dnmt3a-mutant cells is mediated through TNFR2. Targeting TNFR1 may additionally provide the benefit of boosting adaptive immune function through TNF-TNFR2 signaling, as lack of TNFR1-mediated TNFα clearance can lead to increased ligand availability for TNFR2. Our work suggests that TNFR1 blockade strategies, such as humanized antibodies that have been shown to have efficacy in inflammatory disease models, may be useful in individuals with CH that are at high risk of progression to myeloid malignancy. Together, our work suggests that independently manipulating clone fitness and lineage output is possible, which broadens the scope and potential of therapeutic strategies to modulate favorable and unfavorable CH outcomes.

### Example 3. Dnmt3a-Mutant Hematopoietic Stem Cell (HSC) Expansion in the Aged BM Microenvironment is Mediated by Oncostatin M (OSM) Signaling

The mouse model of Dnmt3a-mutant clonal hematopoiesis (Loberg et al., Leukemia 2019) was used to ask what the functional impact of the aging bone marrow microenvironment would be on the selective advantage of mutant cells. The same donor cell source was transplanted into young and aged recipient mice, to place the cells in a young vs. aged bone marrow microenvironment (FIG. 9A). At the end of the transplant experiment, it was found that the proportion and total number of Dnmt3a-mutant hematopoietic stem and progenitor cells (HSPCs) was increased specifically in aged recipient mice (FIG. 9B). This suggests that the factor(s) in the aged bone marrow microenvironment provide an environment where Dnmt3a-mutant HSPCs have a selective advantage.

To determine which factor(s) might be elevated in the aged bone marrow microenvironment that would impact Dnmt3a-mutant HSPCs, donor-derived hematopoietic stem cells (HSCs) were re-isolated from the experiment shown in FIG. 9A (design shown in FIG. 10A). By RNA-seq it was found that Dnmt3a-mutant HSCs had a greater number of differentially expressed genes (both up- and down-regulated) in an aged environment rather than in a young environment (FIG. 10B). Using a bioinformatic tool called Ingenuity Pathway Analysis, candidate upstream factor(s) that would cause these differential gene expression changes were predicted (FIG. 10C). Oncostatin M (OSM) was one of the top identified factors. Based on gene expression patterns, OSM signaling is increased in Dnmt3a-mutant vs. wild-type HSCs, specifically in the context of aged recipient mice (FIG. 10D). OSM levels are elevated in aging locally in the bone marrow compartment, where HSCs reside (FIG. 10E), accounting for why this signature emerges in aged recipients and not in young recipient mice. As several downstream members of the OSM/OSMR signaling pathway were found to be increased in expression in Dnmt3a-mutant HSCs (FIG. 10F), activation by phosphorylation of STAT3 was measured. Data showed that Dnmt3a-mutant HSPCs have greater phosphorylation of STAT3 in response to OSM compared to control HSPCs and this was not a general response as phosphorylation of STAT5 was not seen (FIG. 10G). Together, this data suggests that OSM is elevated in the aged bone marrow microenvironment and that Dnmt3a-mutant HSPCs have a greater signaling response to this factor compared to wild-type HSPCs.

Next, to determine if OSM stimulation has a differential functional effect on Dnmt3a-mutant vs. control HSCs, we established mixed cultures of purified control and Dnmt3a-mutant HSCs in culture conditions that promote HSC self-renewal and expansion. To test HSC function, we transplanted cells at the end of the culture period into recipient mice and measured their engraftment capacity (FIG. 11A). We find that in absence of OSM there is similar or equal engraftment of wild-type and Dnmt3a-mutant competitor cells in recipient mice (FIGS. 11B-11C). In contrast, addition of recombinant OSM to these cultures entirely favors growth of Dnmt3a-mutant HSCs over wild-type cells in the peripheral blood and bone marrow of recipient mice (FIGS. 11B-11C). This finding supports that OSM is a major factor causing clonal hematopoiesis.

To test our hypothesis that OSM signaling is utilized by Dnmt3a-mutant HSCs to maintain their self-renewal and engraftment ability, we assessed the extent to which loss of OSM-OSMR signaling would impact HSPC differentiation. We mixed Dnmt3a-mutant and wild-type HSPCs on stroma cells to promote their differentiation (FIG. 12A). We find that Dnmt3a-mutant HSPCs produce fewer mature myeloid (CD11b+ Gr1+) cells in these cultures compared to wild-type HSPCs (FIG. 12B). We observed that knockout of OSMR on Dnmt3a-mutant HSPCs resulted in a rescue of this phenotype and even greater differentiation and production of mature myeloid cells. This result suggests that OSM-OSMR signaling is utilized by Dnmt3a-mutant HSPCs to preserve their primitive state, and loss of this pathway results in terminal differentiation of these cells.

Together, this data suggests that targeting levels of OSM, or OSM signaling through OSMR or downstream factors, is a valuable approach to treating clonal hematopoiesis and reducing risk of blood cancer.

### Materials and Methods

### Experimental Animals

C57BL/6J (The Jackson Laboratory (JAX) stock #00664, referred to as “CD45.2⁺”) and B6.SJL-Ptprca Pepcb/BoyJ (JAX stock #002014, referred to as “CD45.1⁺”) mice were obtained from, and aged within, JAX. Dnmt3a^{fl-R878H/+} mice (JAX stock #032289) were crossed to B6.CgTg(Mx1-cre)1Cgn/J mice (referred to as Mx-Cre) (JAX stock #003556) or C57BL/6N-Fgd5^{tm3(cre/ERT2)Djr}/J (referred to as Fgd5-Cre) (JAX stock #027789). B6.129S-Tnfrsf1b^{tml/mx}; Tnfrsf1a^{tml/mx} (JAX stock #003243) were crossed to Dnmt3a^{fl-R878H/+}; Mx-Cre. BM from germline Dnmt3a^{+/-} and control wild-type mice were provided by Dr. Challen. The Jackson Laboratory's Institutional Animal Care and Use Committee (IACUC) approved all experiments. All genotypes of mice carrying the Mx-Cre allele were given poly(I:C) every other day for a total of five doses between 2-4 months of age prior to transplant, except where noted below.

### Flow Cytometry and Cell Sorting

Single cell suspensions of BM were prepared by filtering crushed, pooled femurs, tibiae, and iliac crests from each mouse. BM mononuclear cells (MNCs) were isolated by Ficoll-Paque (GE Healthcare Life Sciences) density centrifugation and stained with a combination of fluorochrome-conjugated antibodies from eBioscience, BD Biosciences, or BioLegend: CD45.1 (clone A20), CD45.2 (clone 104), c-Kit (clone 2B8), Sca-1 (clone 108129), CD150 (clone TC15-12F12.2), CD48 (clone HM48-1), FLT3 (Clone A2F10), CD34 (clone RAM34), FcgR (clone 2.4G2), mature lineage (Lin) marker mix and a viability stain. Stained cells were sorted using a FACSAria or a FACSymphony S6 Sorter (BD Biosciences), or analyzed on a FACSymphony A5 or LSR II with Diva software (BD Biosciences). Surface marker profiles are not shown in the present disclosure. PB samples were stained and analyzed using a cocktail of CD45.1, CD45.2, CD11b (clone M1/70), B220 (clone RA3-6B2), CD3e (clone 145-2C11), Ly6g (clone 1A8), and Ly6c (clone HK1.4) on an LSRII (BD). Gating analysis was performed using FlowJo software v10.

Transplants into Young and Aged Recipient Mice

Two months post-poly(I:C), 2×10⁶ post-ficoll whole bone marrow cells from Dnmt3a^{+/+} Mx-Cre or Dnmt3a^{fl-R878H/+} Mx-Cre donors were transplanted into lethally irradiated (10 Gy) young (2-4 mos) or middle-aged (13-15 mos) CD45.1⁺ recipient mice. All transplant recipient mice were monitored every four weeks post-transplant by flow cytometry analysis of PB and were harvested for bone marrow analysis at 40 weeks post-transplant.

### Polyvinyl Alcohol (PVA) Culture and Transplants

Two months post-poly(I:C), 25 CD45.2⁺ from Dnmt3a^{+/+} Mx-Cre or Dnmt3a^{fl-R878H/+} Mx-Cre donors (CD45.2+) and 25 CD45.1⁺/CD45.2⁺ HSCs were sorted into a 96-well plate with Ham's F12 media containing final concentrations of 1× Penicillin-streptomycin-glutamine (Gibco cat. #10378-016), 10 mM HEPES (Gibco cat. #15630080), 1× Insulin-transferrin-selenium-ethanolamine (Gibco cat. #51500-056), 100 ng/mL recombinant murine TPO (Biolegend cat. #593302), 10 ng/mL recombinant murine SCF (StemCell Technologies cat. #78064), and 1 mg/mL polyvinyl alcohol (Sigma cat. #P8136)¹⁹+/−10 ng/mL recombinant murine TNF-α (PeproTech cat. #315-01A) and cultured for seven days at 37° C. and 5% CO₂. TNF-α was spiked into the cultures on day 4 and 6. On day 7, half of the wells were stained and analyzed by flow cytometry and half of the wells were harvested, mixed with 1×10⁶ CD45.1⁺ post-ficoll whole BM cells, and transplanted into young, lethally irradiated CD45.1⁺ recipients. PB was tracked monthly for six months post-transplant via flow cytometry.

### Etanercept Transplants

1×10⁶ BM cells from 2-4-month-old Dnmt3a^{+/+} Mx-Cre or Dnmt3a^{fl-R878H/+} Mx-Cre donors were competitively transplanted with wild-type CD45.1+ CD45.2+ F1 BM cells in 2-4-month-old CD45.1+ lethally irradiated recipients. Recipients were allowed to recover for one month and then poly(I:C) was administered every other day for a total of five injections to induce Cre expression. 28 weeks post-poly(I:C), bone marrow was harvested and 5×10⁶ whole bone marrow cells were transplanted into 2-4-month-old lethally irradiated CD45.1+ recipients. 24 weeks post-secondary transplant, etanercept (25 mg/kg, Millipore Sigma #Y0001969) or PBS was administered via IP twice per week for four weeks. PB was monitored weekly and at 28 weeks post-transplant, bone marrow was harvested for analysis.

### TNFR Knockout Transplants

1×10⁶ CD45.2+ cells were competed against 1×10⁶ CD45.1+ whole BM cells and transplanted into aged, lethally irradiated CD45.1+ recipient animals. One-month post-transplant, recipients received one IP injection of poly(I:C) and recombination was checked via PCR on PB. One month post poly(I:C), animals were bled monthly for 16 weeks. Bone marrow was harvested and 4×10⁶ whole BM cells were used for secondary transplantation into aged, lethally irradiated recipients. PB was analyzed starting at one-month post-transplant and continued monthly for 20 weeks. BM was harvested and analyzed by flow cytometry and Lin-c-kit+ CD45.2+ cells were FACS-sorted for single-cell RNA-sequencing. Complete blood counts (CBC) were performed on a Advia 120 Hematology Analyzer (Siemens).

### Bulk RNA-Sequencing and Analysis

One-month post-poly(I:C), 1×10⁶ whole BM cells from Dnmt3a^{+/+} Mx-Cre or Dnmt3a^{R878H/+} Mx-Cre donors were transplanted into sublethally irradiated (6 Gy) young (2 mos) or middle-aged (13-15 mos) CD45.1+ recipient mice. Recipients were harvested at four months post-transplant for PB and BM analysis. CD45.2+ HSCs were sorted directly into RLT buffer (Qiagen) and flash frozen. Total RNA was isolated using the RNAeasy Micro Kit (Qiagen) including DNase treatment, and sample quality was assessed using a Nanodrop 2000 spectrophotometer (Thermo Scientific) and RNA 6000 Pico LabChip assay (Agilent Technologies). Libraries were prepared using the Ovation RNA-seq System V2 (NuGen) and Hyper Prep Kit (Kapa Biosystems). Library quality and concentration evaluated using D5000 ScreenTape assay (Agilent) and quantitative PCR (Kapa Biosystems). Libraries were pooled and sequenced 75 bp single end on the NextSeq (Illumina) using NextSeq High Output Kit v2 reagents at a sequencing depth of >30 million reads per sample. Trimmed alignment files were processed using RSEM (v1.2.12). Alignment was completed using Bowtie 2 (v2.2.0). Expected read counts per gene produced by RSEM were rounded to integer values, filtered to include only genes that have at least two samples within a sample group having a cpm>1, and were passed to edgeR (v3.14.0) for differential expression analysis. The GLM likelihood ratio test was used for differential expression in pairwise comparisons between sample groups which produced exact p-values per test. The Benjamini and Hochberg's algorithm (p-value adjustment) was used to control the false discovery rate (FDR). Features with FDR-adjusted p-value<0.05 were declared significantly differentially expressed. Differentially expressed genes were investigated for overlap with published datasets using Gene Set Enrichment Analysis (GSEA) and upstream regulators were predicted using Ingenuity Pathway Analysis (IPA) software (Qiagen).

### Single Cell RNA Sequencing and Analysis

Cells were counted on a Countess II automated cell counter (ThermoFisher) and 12,000 cells were loaded on to one lane of a 10× Chromium microfluidic chip. (10× Genomics). Single cell capture, barcoding and library preparation were performed using the 10× Chromium version 3.1 chemistry, according to the manufacturer's protocol (#CG000315). cDNA and libraries were checked for quality on Agilent 4200 Tapestation and quantified by KAPA qPCR before sequencing; each gene expression library was sequenced at 18.75% of an Illumina NovaSeq 6000 S4 flow cell lane, targeting 6,000 barcoded cells with an average sequencing depth of 75,000 reads per cell. Illumina base call files for all libraries were demultiplexed and converted to FASTQs using bcl2fastq v2.20.0.422 (Illumina). The Cellranger pipeline (10× Genomics, version 6.0.0) was used to align reads to the mouse reference GRCm38.p93 (mm10 10× Genomics reference 2020-A), de-duplicate reads, call cells, and generate cell by gene digital counts matrices for each library. The resultant counts matrices were uploaded into PartekFlow (version 10.0.22.0428) for downstream analysis and visualization. This included log transformation of count data, principal component analysis, graph-based clustering from the top 20 principal components using the Louvain Algorithm, UMAP visualization, and pathway enrichment analysis. Trajectory and pseudotime analysis were performed using Monocle 3.

### Statistical Analysis

No sample group randomization or blinding was performed. All statistical tests, including evaluation of normal distribution of data and examination of variance between groups being statistically compared, were assessed using Prism 9 software (GraphPad).

### Data Availability

All data in this study are deposited in the NCBI Gene Expression Omnibus (GEO) under accession number GSE189406 (bulk RNA-seq) and GSE203550 (single-cell RNA-seq)

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A method of treating clonal hematopoiesis in a subject in need thereof, comprising:
administering to the subject a mitochondria-targeted antioxidant in an amount effective to suppress clonal hematopoiesis in the subject, relative to an untreated control.

2. The method of claim 1, wherein the subject exhibits signs of defects in mitochondrial metabolism.

3. The method of claim 2, wherein administration of the mitochondria-targeted antioxidant improves mitochondrial metabolism in the subject.

4. The method of any one of claim 1-3, wherein the mitochondria-targeted antioxidant is a shortened form of the antioxidant ubiquinol with triphenylphosphonium (e.g., MitoQ®).

5. The method of any one of claims 1-4, further comprising identifying the subject as exhibiting a sign of clonal hematopoiesis.

6. The method of claim 5, wherein the sign of clonal hematopoiesis is a defect in mitochondrial metabolism.

7. A method of treating clonal hematopoiesis in a subject in need thereof, comprising:
administering to the subject a TNF signaling inhibitor in an amount effective to suppress clonal hematopoiesis in the subject, relative to an untreated control.

8. The method of claim 7, wherein the subject exhibits signs of elevated TNF signaling.

9. The method of claim 8, wherein administration of the TNF signaling inhibitor specifically reduces TNF signaling through TNFR1 in the subject.

10. The method of any one of claims 7-9, wherein the TNF signaling inhibitor is an antibody that specifically binds to TNFR1.

11. The method of any one of claims 7-10, further comprising identifying the subject as exhibiting a sign of clonal hematopoiesis.

12. The method of claim 11, wherein the sign of clonal hematopoiesis is elevated TNF signaling.

13. A method of treating clonal hematopoiesis in a subject in need thereof, comprising:
administering to the subject a OSM signaling inhibitor in an amount effective to suppress clonal hematopoiesis in the subject, relative to an untreated control.

14. The method of claim 13, wherein the subject exhibits signs of elevated OSM signaling.

15. The method of claim 14, wherein administration of the OSM signaling inhibitor reduces OSM signaling in the subject.

16. The method of any one of claims 13-15, wherein the OSM signaling inhibitor is an antibody that specifically binds to OSM or OSMR.

17. The method of any one of claims 13-16, further comprising identifying the subject as exhibiting a sign of clonal hematopoiesis.

18. The method of claim 17, wherein the sign of clonal hematopoiesis is elevated OSM signaling.

19. The method of any one of the preceding claims, wherein the subject is at risk of blood cancer.

20. The method of any one of the preceding claims, wherein the subject is at risk of cardiovascular disease.
